(19) United States
(12) Patent Application Publication
(10) Pub. No.: US 2025/0304598 A1
(43) Pub. Date: Oct. 2, 2025

(54) ORGANIC ELECTRO-OPTIC CHROMOPHORES

(71) Applicant: University of Washington, Seattle, WA (US)

(72) Inventors: Delwin Elder, Seattle, WA (US); Lewis Johnson, Seattle, WA (US); Bruce Robinson, Seattle, WA (US); Huajun Xu, Seattle, WA (US)

(73) Assignee: University of Washington, Seattle, WA (US)

(21) Appl. No.: 19/239,483

(22) Filed: Jun. 16, 2025

Related U.S. Application Data
(63) Continuation of application No. 18/304,764, filed on Apr. 21, 2023, now Pat. No. 12,371,440.
Application No. 18/304,764 is a continuation of application No. 17/713,055, filed on Apr. 4, 2022, now Pat. No. 11,634,429.
Application No. 17/713,055 is a continuation of application No. PCT/US2020/054081, filed on Oct. 2, 2020.
(60) Provisional application No. 62/911,067, filed on Oct. 4, 2019.
(60) Provisional application No. 62/934,398, filed on Nov. 12, 2019.

(51) Int. Cl. C07D 495/04; C07D 307/28; C07F 7/08; G02F 1/361; H10K 85/40; H10K 85/60

(52) U.S. Cl. CPC C07D 495/04; C07D 307/28; C07F 7/0812; G02F 1/3613; G02F 1/3614; H10K 85/40; H10K 85/636; H10K 85/655; H10K 85/6576

(57) ABSTRACT

Chromophores with large hyperpolarizabilities, films with electro-optic activity comprising the chromophores, and electro-optic devices comprising the chromophores are disclosed.

### CROSS-REFERENCES TO RELATED APPLICATIONS

This application is a continuation of U.S. patent application Ser. No. 18/304,764, filed Apr. 21, 2023, which is a continuation of U.S. patent application Ser. No. 17/713,055, filed Apr. 4, 2022, which is a continuation of International Application No. PCT/US2020/054081, filed Oct. 2, 2020, which claims the benefit of U.S. Provisional Application No. 62/911,067, filed Oct. 4, 2019, and U.S. Provisional Application No. 62/934,398, filed Nov. 12, 2019, the disclosures of which are incorporated herein by reference in their entirety.

### STATEMENT OF GOVERNMENT LICENSE RIGHTS

This invention was invention was made with government support under Grant No. FA9550-15-1-0319, awarded by the Air Force Office of Scientific Research and Grant No. DMR1303080, awarded by the National Science Foundation. The government has certain rights in the invention.

### FIELD OF THE INVENTION

The present disclosure provides chromophores useful for inclusion in films having electro-optic activity and electro-optic devices.

### BACKGROUND

Organic electro-optic (OEO) materials have recently seen a resurgence in interest due to the development of silicon-organic hybrid (SOH) and plasmonic-organic hybrid (POH) devices, which enable combining the high intrinsic electro-optic activity of certain classes of organic chromophores with small device size and potential for chip-scale integration with CMOS electronics. The size of electro-optic devices is proportional to the voltage-length product U Lμ1/n³r₃₃, where U_{π} is the voltage required to induce a phase shift of π over a path length L, n is the refractive index of the electro-optic material, and r₃₃ is the electro-optic coefficient of the material. In an OEO material, r₃₃μ_{N} [symbol] cos³ [symbol] , where ρ_{N} is the number density (concentration) of chromophores that possess a large molecular hyperpolarizability (β), and have been aligned such that their dipole moments are acentrically ordered (nonzero [symbol] cos³ [symbol] ), where θ is the angle between the dipole moments of the chromophores and the axis normal to the electrodes of the electro-optic device.

High hyperpolarizability chromophores typically have a donor-π bridge-acceptor (D-π-A) structure, containing an electron donating moiety such as a substituted amine group, an electron-accepting moiety containing strong electron-withdrawing groups such as cyano (CN) or nitro (NO₂), connected by a π-conjugated linker, often containing ene/polyene and/or heteroaromatic groups, such as a D-π-A chromophore, known as JRD1 depicted in FIG. 1.

A need exists for chromophores having higher chromophore hyperpolarizabilities and favorable stability and processability, enabling higher r₃₃ and smaller U_{π}L in devices for any given chromophore concentration.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a compound of Formula A:

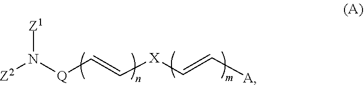

wherein:

A is a π-electron acceptor group;

X is:

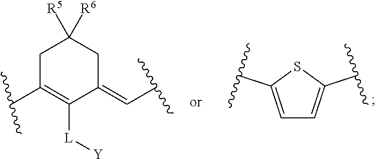

L is absent or L is S or O;

Y is H, optionally substituted C1-C20 alkyl, optionally substituted C3-C50 heteroalkyl, optionally substituted C3-C10 cycloalkyl, optionally substituted C3-C10 cycloheteroalkyl;

n is 1, 2, or 3;

n is 1, 2, or 3, and

R⁵ and R⁶ are independently H, optionally substituted C1-C10 alkyl, optionally substituted C3-C10 heteroalkyl, optionally substituted C3-C10 cycloalkyl, or optionally substituted C3-C10 cycloheteroalkyl;

Q is

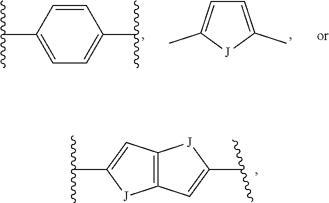

J, at each occasion, is independently S, O, or NR⁸;

R⁸ is H, optionally substituted C1-C10 alkyl, optionally substituted C3-C10 heteroalkyl, optionally substituted C3-C10 cycloalkyl, optionally substituted C3-C10 cycloheteroalkyl, optionally substituted C6-C10 aryl, or optionally substituted C5-C10 heteroaryl; and

when Q is

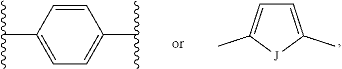

Z¹ is optionally substituted C6-C10 aryl, or optionally substituted C5-C10 heteroaryl and Z² is H, optionally substituted C1-C10 alkyl, optionally substituted C3-C10 heteroalkyl, optionally substituted C3-C10 cycloalkyl, optionally substituted C3-C10 cycloheteroalkyl, optionally substituted C6-C10 aryl, or optionally substituted C5-C10 heteroaryl or

when Q is

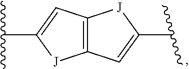

Z¹ and Z² are independently H, optionally substituted C1-C10 alkyl, optionally substituted C3-C10 heteroalkyl, optionally substituted C3-C10 cycloalkyl, optionally substituted C3-C10 cycloheteroalkyl, optionally substituted C6-C10 aryl, or optionally substituted C5-C10 heteroaryl.

In some embodiments, the compound is represented by Formula A1:

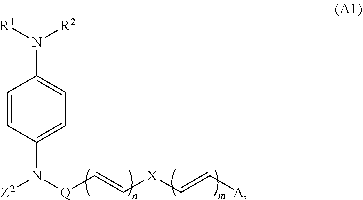

wherein:

Z2, Q, X, A, n, and m are as defined above; and

R¹ and R² are independently H, optionally substituted C1-C10 alkyl, optionally substituted C3-C10 heteroalkyl, optionally substituted C3-C10 cycloalkyl, or optionally substituted C3-C10 cycloheteroalkyl.

In some embodiments, the compound is represented by Formula A2:

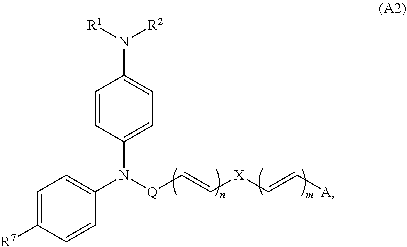

wherein:

Q, X, A, n, and m are as defined above;

R¹ and R² are independently H, optionally substituted C1-C10 alkyl, optionally substituted C3-C10 heteroalkyl, optionally substituted C3-C10 cycloalkyl, or optionally substituted C3-C10 cycloheteroalkyl;

R⁷ is H, optionally substituted C1-C10 alkyl, optionally substituted C3-C10 heteroalkyl, optionally substituted C3-C10 cycloalkyl, or optionally substituted C3-C10 cycloheteroalkyl, optionally substituted C1-C10 alkyloxy, optionally substituted C3-C10 heteroalkyloxy, or NR³R⁴; and

R³ and R⁴ are independently H, optionally substituted C1-C10 alkyl, optionally substituted C3-C10 heteroalkyl, optionally substituted C3-C10 cycloalkyl, or optionally substituted C3-C10 cycloheteroalkyl.

In some embodiments, the compound is represented by Formula A3:

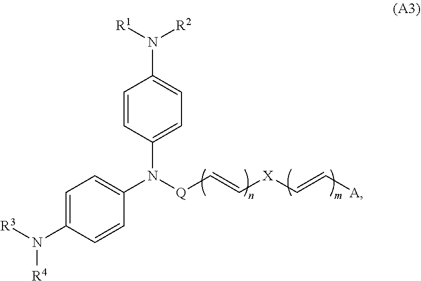

wherein:

Q, X, A, n, and m are as defined above;

R¹ and R² are independently H, optionally substituted C1-C10 alkyl, optionally substituted C3-C10 heteroalkyl, optionally substituted C3-C10 cycloalkyl, or optionally substituted C3-C10 cycloheteroalkyl; and

R³ and R⁴ are independently H, optionally substituted C1-C10 alkyl, optionally substituted C3-C10 heteroalkyl, optionally substituted C3-C10 cycloalkyl, or optionally substituted C3-C10 cycloheteroalkyl.

In some embodiments, the compound is represented by Formula A4:

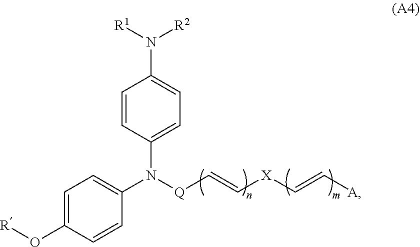

wherein:

Q, X, A, n, and m are as defined above;

R¹ and R² are independently H, optionally substituted C1-C10 alkyl, optionally substituted C3-C10 heteroalkyl, optionally substituted C3-C10 cycloalkyl, or optionally substituted C3-C10 cycloheteroalkyl; and

R′ is optionally substituted C1-C10 alkyl, optionally substituted C3-C10 heteroalkyl, optionally substituted C3-C10 cycloalkyl, or optionally substituted C3-C10 cycloheteroalkyl.

In some embodiments, Q is:

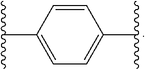 [SMILES: *c1ccc(*)cc1]

In some embodiments, Q is:

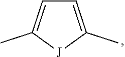

wherein J is i S, O, or NR⁸; and R⁸ is H, optionally substituted C1-C10 alkyl, optionally substituted C3-C10 heteroalkyl, optionally substituted C3-C10 cycloalkyl, optionally substituted C3-C10 cycloheteroalkyl, optionally substituted C6-C10 aryl, or optionally substituted C5-C10 heteroaryl. In some embodiments, Q is:

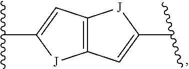

wherein J, at each occasion, is independently S, O, or NR⁸; and

R⁸ is H, optionally substituted C1-C10 alkyl, optionally substituted C3-C10 heteroalkyl, optionally substituted C3-C10 cycloalkyl, optionally substituted C3-C10 cycloheteroalkyl, optionally substituted C6-C10 aryl, or optionally substituted C5-C10 heteroaryl.

In some embodiments, J is S.

In some embodiments, A is

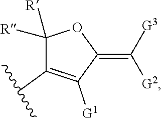

wherein R′ and R″ are independently selected from optionally substituted C1-C12 alkyl (e.g., fluorinated alkyl) and optionally substituted C6-C10 aryl (e.g., fluorinated aryl), and G¹, G², and G³ are independently selected from F, CN, CF₃, SO₂CF₃.

In some embodiments, A is

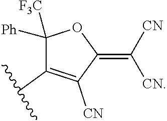 [SMILES: *C1=C(C#N)C(=C(C#N)C#N)OC1(c1ccccc1)C(F)(F)F]

In some embodiments, m is 1. In some embodiments, n is 1.

In some embodiments, the compound is a compound of Formula A5:

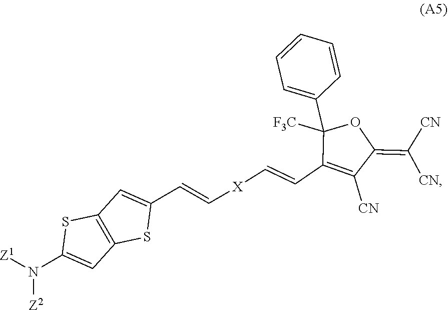

wherein:

Z¹ and Z² are independently H, optionally substituted C1-C10 alkyl, optionally substituted C3-C10 heteroalkyl, optionally substituted C3-C10 cycloalkyl, optionally substituted C3-C10 cycloheteroalkyl, optionally substituted C6-C10 aryl, or optionally substituted C5-C10 heteroaryl;

X is:

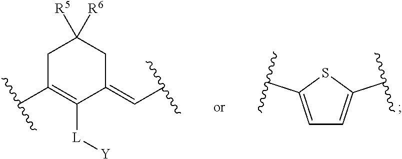

L is absent or L is S or O;

Y is H, optionally substituted C1-C20 alkyl, optionally substituted C3-C50 heteroalkyl, optionally substituted C3-C10 cycloalkyl, or optionally substituted C3-C10 cycloheteroalkyl; and

R⁵ and R⁶ are independently H, optionally substituted C1-C10 alkyl, optionally substituted C3-C10 heteroalkyl, optionally substituted C3-C10 cycloalkyl, or optionally substituted C3-C10 cycloheteroalkyl.

In some embodiments, the compound is a compound of Formula A6:

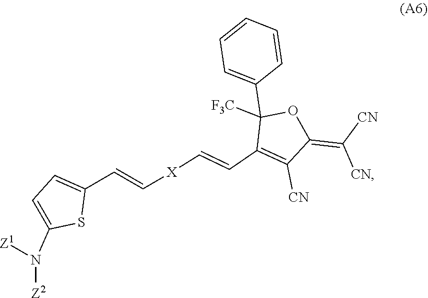

wherein:

Z¹ is optionally substituted C6-C10 aryl or optionally substituted C5-C10 heteroaryl; Z² is H, optionally substituted C1-C10 alkyl, optionally substituted C3-C10 heteroalkyl, optionally substituted C3-C10 cycloalkyl, optionally substituted C3-C10 cycloheteroalkyl, optionally substituted C6-C10 aryl, or optionally substituted C5-C10 heteroaryl;

X is:

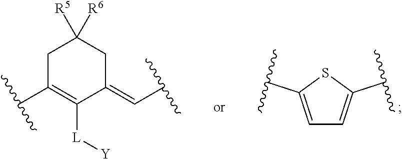

L is absent or L is S or O;

Y is H, optionally substituted C1-C20 alkyl, optionally substituted C3-C50 heteroalkyl, optionally substituted C3-C10 cycloalkyl, or optionally substituted C3-C10 cycloheteroalkyl; and

R⁵ and R⁶ are independently H, optionally substituted C1-C10 alkyl, optionally substituted C3-C10 heteroalkyl, optionally substituted C3-C10 cycloalkyl, or optionally substituted C3-C10 cycloheteroalkyl.

In some embodiments, the compound is a compound of Formula A7:

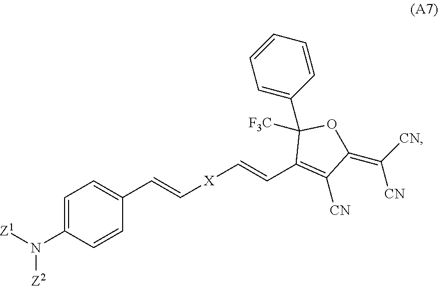

wherein:

Z¹ is optionally substituted C6-C10 aryl or optionally substituted C5-C10 heteroaryl;

Z² is H, optionally substituted C1-C10 alkyl, optionally substituted C3-C10 heteroalkyl, optionally substituted C3-C10 cycloalkyl, optionally substituted C3-C10 cycloheteroalkyl, optionally substituted C6-C10 aryl, or optionally substituted C5-C10 heteroaryl;

X is:

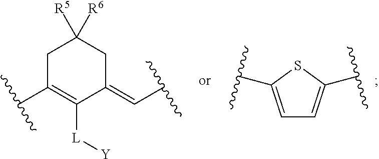

L is absent or L is S or O;

Y is H, optionally substituted C1-C20 alkyl, optionally substituted C3-C50 heteroalkyl, optionally substituted C3-C10 cycloalkyl, or optionally substituted C3-C10 cycloheteroalkyl; and

R⁵ and R⁶ are independently H, optionally substituted C1-C10 alkyl, optionally substituted C3-C10 heteroalkyl, optionally substituted C3-C10 cycloalkyl, or optionally substituted C3-C10 cycloheteroalkyl.

In some embodiments, the compound comprises one or more reactive groups that can form a covalent bond when reacted with a counterpart reactive group. In some embodiments, the one or more reactive groups is a group crosslinkable by (4+2) cycloaddition.

In some embodiments, at least one of Z¹ and X is substituted with a group selected from

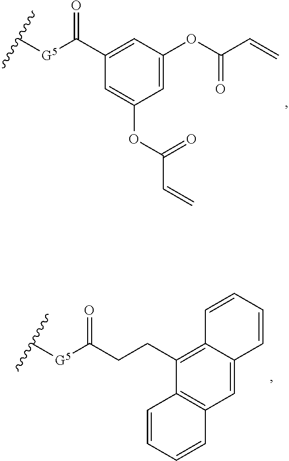

and OSiR¹⁰R¹¹R¹², wherein G⁵ is NH, O, S, or N(C1-C10-alkyl) and R¹⁰, R¹¹, and R¹² are independently H, optionally substituted C1-C10 alkyl, or optionally substituted C6-C10 aryl.

In some embodiments, L-Y is H, OL¹OSiR¹⁰R¹¹R¹², or SL¹OSiR¹⁰R¹¹R¹², wherein L¹ is an optionally substituted C2-C20 alkylene or optionally substituted C3-C50 heteroalkylene; and R¹⁰, R¹¹, and R¹² are independently H, optionally substituted C1-C10 alkyl, or optionally substituted C6-C10 aryl.

In some embodiments, X is:

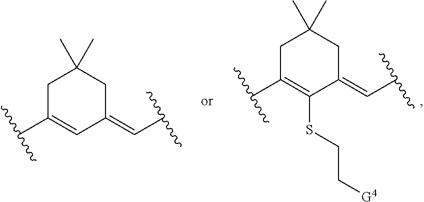

wherein G⁴ is OSiR¹⁰R¹¹R¹², wherein R¹⁰, R¹¹, and R¹² are independently H, optionally substituted C1-C10 alkyl, or optionally substituted C6-C10 aryl, or G⁴ is

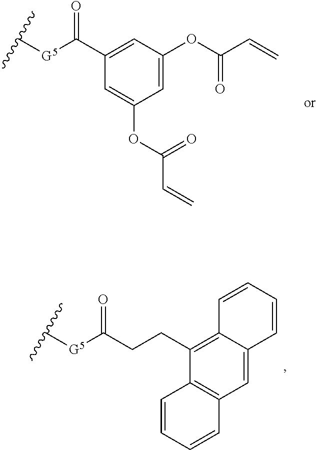

wherein G⁵ is NH, O, S, or N(C1-C10-alkyl).

In some embodiments, the compound is a compound of Formula A8:

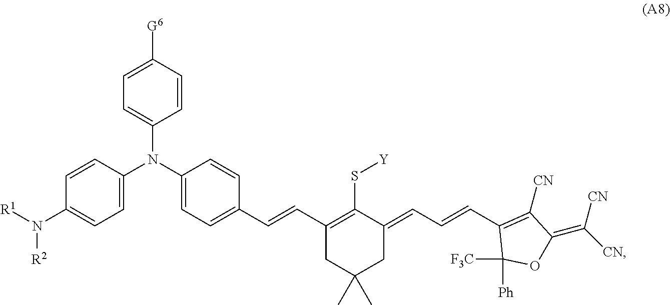

wherein:

G⁶ is OR′ or NR′R″, wherein R′ and R″ are independently optionally substituted C1-C10 alkyl;

R¹ is H or an optionally substituted C1-C10 alkyl,

R² is optionally substituted C1-C10 alkyl, optionally substituted C3-C10 heteroalkyl, optionally substituted C3-C10 cycloalkyl, or optionally substituted C3-C10 cycloheteroalkyl; and

Y is optionally substituted C1-C10 alkyl, optionally substituted C3-C10 heteroalkyl, optionally substituted C3-C10 cycloalkyl, or optionally substituted C3-C10 cycloheteroalkyl; and

wherein at least one of R², Y, and G⁶ is substituted with a group selected from

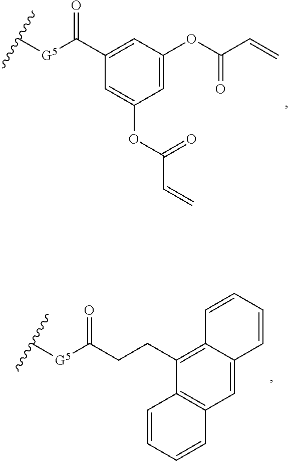

and OSiR¹⁰R¹¹R¹²,

wherein G⁵ is NH, O, S, or N(C1-C10-alkyl) and R¹⁰, R¹¹, and R¹² are independently H, optionally substituted C1-C10 alkyl, or optionally substituted C6-C10 aryl.

In some embodiments, the compound is Compound I, Compound II, Compound III, or Compound IV, Compound V, Compound VI, Compound VII, Compound VIII, Compound IX, Compound X, Compound XI, Compound XII, Compound XIII, Compound XIV, or Compound XV depicted below.

In another aspect, the disclosure provides a film having electro-optic activity comprising one or more compounds disclosed herein. In some embodiments, the film further comprises a polymer. In some embodiments, the polymer is polymethylmethacrylate (PMMA). In some embodiments, the film has an r₃₃ value of greater than about 100 pm/V. In some embodiments, the film has an r₃₃ value of greater than about 1000 pm/V. In some embodiments, the film has a T_{g} of about 105° C. or greater.

In another aspect, the disclosure provides a method for forming a film having electro-optic activity, comprising depositing a compound or mixture containing a compound of disclosed herein on a substrate to provide a film, applying an aligning force to the film at a temperature sufficient to provide a film having at least a portion of the compounds aligned, and reducing the temperature of the film to provide a film having electro-optic activity.

In another aspect, the disclosure provides an electro-optic device comprising a compound of disclosed herein.

In another aspect, the disclosure provides an electro-optic device comprising a film disclosed herein.

In some embodiments, the electro-optic device further comprises one or more charge blocking layers. In some embodiments, the one or more charge blocking layers comprises poly (benzocyclobutene) (BCB), TiO₂, MoO₃, ZrO₂, HfO₂, SiO₂, Al₂O₃, Si₃N₄, or a combination thereof. In some embodiments, the device is an electro-optic modulator, antenna, Mach-Zehnder modulator, phase modulator, silicon-organic hybrid modulator, plasmonic-organic hybrid modulator, electrical-to-optical convertor, terahertz detector, frequency shifter, or frequency comb source.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 depicts known JRD1 chromophore, with donor, π-bridge, and acceptor labeled. JRD1 contains a substituted aniline donor, a ring-locked polyene bridge, and a CF₃-phenyl substituted tricyanofuran (TCF) acceptor

FIG. 2 shows the synthesis of an exemplary chromophore Compound IV.

FIGS. 3A and 3B depicts real (n, FIG. 3A) and imaginary (k, FIG. 3B) (components of the refractive index for exemplary chromophores Compound I, Compound II, Compound III, or Compound IV.

FIGS. 4A and 4B shows real (n, FIG. 4A) and imaginary (k, FIG. 4B) components of the refractive index for 10 wt % and 25 wt % concentrations of exemplary Compound II and 25 wt % concentration of exemplary Compound IV compared with 25 wt % JRD1 in PMMA.

FIGS. 5A-5D show electro-optic measurements on thin films for exemplary Compound II (5A and 5B) and exemplary Compound IV (5C and 5D) in PMMA obtained at 1310 nm using the Teng-man ellipsometry method.

FIGS. 6A and 6B depicts the poling efficiency (electro-optic coefficient as a function of poling field) for exemplary Compound VI under various conditions (FIG. 6A) and the real (n) and imaginary (k) components of the refractive index for exemplary Compound VI (FIG. 6B).

FIG. 7 shows the synthesis of an exemplary chromophore Compound VI.

### DETAILED DESCRIPTION

Provided herein are chromophores with large hyperpolarizabilities, films with electro-optic activity comprising the chromophores, and electro-optic devices comprising the chromophores. The chromophores of the disclosure possess a large molecular hyperpolarizability, which enables a large electro-optic coefficient (r₃₃) in films containing the chromophores.

The polarizable chromophore compounds or polarizable chromophores disclosed herein are second-order nonlinear optical chromophore compounds. As used herein, the term “chromophore” refers to a compound that can absorb light in the visible spectral range and is colored. The terms “polarizable chromophore compound,” “polarizable chromophore,” and “chromophore” are used interchangeably throughout the disclosure, unless indicated otherwise. In the context of the disclosure, the term “nonlinear” refers second order effects that arise from the nature of the polarizable chromophore compound (i.e., “push-pull” chromophore compound) having the general structure D-π-A, where D is an electron donor, A is an electron acceptor, and π is a π-bridge that conjugates the donor to the acceptor.

A “donor” (represented by “D”) is an atom or group of atoms with low electron affinity relative to an acceptor (defined below) such that, when the donor is conjugated to an acceptor through a π-bridge, electron density is transferred from the donor to the acceptor.

An “acceptor” (represented by “A”) is an atom or group of atoms with high electron affinity relative to a donor such that, when the acceptor is conjugated to a donor through a π-bridge, electron density is transferred from the acceptor to the donor.

A “π-bridge” or “conjugated bridge” (represented in chemical structures by π″ or “πₙ” where n is an integer) is comprised of an atom or group of atoms through which electrons can be delocalized from an electron donor (defined above) to an electron acceptor (defined above) through the orbitals of atoms in the bridge. Preferably, the orbitals will be p-orbitals on multiply bonded carbon atoms such as those found in alkenes, alkynes, neutral or charged aromatic rings, and neutral or charged heteroaromatic ring systems. Additionally, the orbitals can be p-orbitals on multiply bonded atoms such as boron or nitrogen or organometallic orbitals. The atoms of the bridge that contain the orbitals through which the electrons are delocalized are referred to here as the “critical atoms.” The number of critical atoms in a bridge can be a number from 1 to about 30. The critical atoms can also be substituted further with the following: “alkyl” as defined below, “aryl” as defined below, or “heteroalkyl” as defined below. One or more atoms, with the exception of hydrogen, on alkyl, aryl, or heteroalkyl substituents of critical atoms in the bridge may be bonded to atoms in other alkyl, aryl, or heteroalkyl substituents to form one or more rings.

In one aspect, provided herein are polarizable chromophore compounds of Formula A:

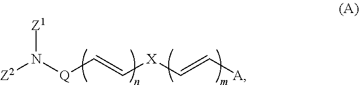

wherein:

A is a π-electron acceptor group;

Z¹ and Z² are independently H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl;

Q is

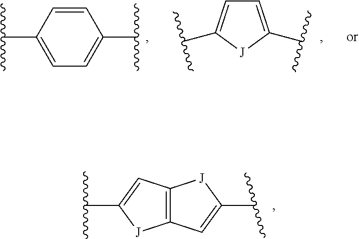

J, at each occasion, is independently S, O, or NR⁸;

R⁸ is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl;

X is:

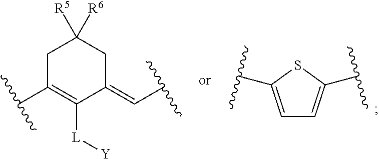

L is absent or L is S or O;

Y is H, optionally substituted C₁-C₂₀ alkyl, optionally substituted C₃-C₅₀ heteroalkyl, optionally substituted C₃-C₁₀ cycloalkyl, or optionally substituted C₃-C₁₀ cycloheteroalkyl;

n is 1, 2, or 3;

n is 1, 2, or 3, and

R⁵ and R⁶ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl,

provided that when

when Q is

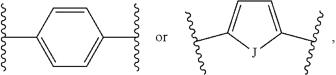

Z¹ is optionally substituted C₆-C₁₀ aryl or optionally substituted C₅-C₁₀ heteroaryl and Z² is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl,

and when Q is

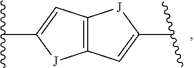

Z¹ and Z² are independently H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl.

As used herein, the terms “alkyl,” “alkenyl,” and “alkynyl” include straight-chain, branched-chain, and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C, as C₁-C₁₀, C-C10, or C1-10.

The terms “heteroalkyl,” “heteroalkenyl,” and “heteroalkynyl,” as used herein, mean the corresponding hydrocarbons wherein one or more chain carbon atoms have been replaced by a heteroatom. Exemplary heteroatoms include N, O, S, and P. When heteroatoms are allowed to replace carbon atoms, for example, in heteroalkyl groups, the numbers describing the group, though still written as e.g. C3-C10, represent the sum of the number of carbon atoms in the cycle or chain and the number of such heteroatoms that are included as replacements for carbon atoms in the cycle or chain being described.

Typically, the alkyl, alkenyl, and alkynyl substituents contain 1-20 carbon atoms (alkyl) or 2-10 carbon atoms (alkenyl or alkynyl). Preferably, they contain 1-10 carbon atoms (alkyl) or 2-10 carbon atoms (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond. As used herein, the terms “cycloalkyl,” “cycloalkenyl,” and “cycloalkynyl” specifically refer to cyclic alkyls, alkenyls, and alkynyls, respectively.

As used herein, the terms “alkylene,” “alkenylene,” and “alkynylene” can include straight-chain, branched-chain, and cyclic divalent hydrocarbyl radicals, and combinations thereof. As used herein, the terms “cycloalkylene,” “cycloalkenylene,” and “cycloalkynylene” specifically refer to cyclic divalent hydrocarbyl radicals.

Alkyl, alkenyl, and alkynyl groups can be optionally substituted to the extent that such substitution makes sense chemically. Typical substituents include, but are not limited to, halogens (F, Cl, Br, I), ═O, ═N—CN, ═N—OR, ═NR, OR, NR₂, SR, SO₂R, SO₂NR₂, NRSO₂R, NRCONR₂, NRC(O)OR, NRC(O)R, CN, C(O)OR, C(O)NR₂, OC(O)R, C(O)R, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halogens (F, Cl, Br, I), ═O, ═N—CN, ═N—OR′, ═NR′, OR′, NR′₂, SR′, SO₂R′, SO₂NR′₂, NR′SO₂R′, NR′CONR′₂, NR′C(O)OR′, NR′C(O)R′, CN, C(O)OR′, C(O)NR′₂, OC(O)R′, C(O)R′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl, and alkynyl groups can also be substituted by C1-C8 acyl, C2-C8 heteroacyl, C6-C10 aryl or C5-C10 heteroaryl, each of which can be substituted by the substituents that are appropriate for the particular group.

While “alkyl” as used herein includes cycloalkyl and cycloalkylalkyl groups, the term “cycloalkyl” is used herein to describe a carbocyclic non-aromatic group that is connected via a ring carbon atom, and “cycloalkylalkyl” is used to describe a carbocyclic non-aromatic group that is connected to the molecule through an alkyl linker. Similarly, “heterocyclyl” is used to identify a non-aromatic cyclic group that contains at least one heteroatom as a ring member and that is connected to the molecule via a ring atom, which may be C or N; and “heterocyclylalkyl” may be used to describe such a group that is connected to another molecule through an alkylene linker. As used herein, these terms also include rings that contain a double bond or two, as long as the ring is not aromatic.

“Aromatic” or “aryl” substituent or moiety refers to a monocyclic or fused bicyclic moiety having the well-known characteristics of aromaticity; examples include phenyl and naphthyl. Similarly, the terms “heteroaromatic” and “heteroaryl” refer to such monocyclic or fused bicyclic ring systems which contain as ring members one or more heteroatoms. Suitable heteroatoms include N, O, and S, inclusion of which permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl, and fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably, the monocyclic heteroaryls contain 5-6 ring members, and the bicyclic heteroaryls contain 8-10 ring members.

Aryl and heteroaryl moieties can be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halogens (F, Cl, Br, I), OR, NR₂, SR, SO₂R, SO₂NR₂, NRSO₂R, NRCONR₂, NRC(O)OR, NRC(O)R, CN, C(O)OR, C(O)NR₂, OC(O)R, C(O)R, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

“Optionally substituted,” as used herein, indicates that the particular group being described may have one or more hydrogen substituents replaced by a non-hydrogen substituent. In some optionally substituted groups or moieties, all hydrogen substituents are replaced by a non-hydrogen substituent, e.g., C1-C6 alkyl, C2-C6 heteroalkyl, alkynyl, halogens (F, Cl, Br, I), N₃, OR, NR₂, SiR₃, OSiR₃, SR, SO₂R, SO₂NR₂, NRSO₂R, NRCONR₂, NRC(O)OR, NRC(O)R, CN, C(O)OR, C(O)NR₂, OC(O)R, C(O)R, oxo, and NO₂, wherein each R is independently H, C1-C6 alkyl, C6-C10 aryl, or C2-C6 heteroalkyl. Where an optional substituent is attached via a double bond, such as a carbonyl oxygen or oxo (═O), the group takes up two available valences, so the total number of substituents that may be included is reduced according to the number of available valences. In some embodiments, the optional non-hydrogen substituent is OSiRR′R″, wherein R, R′, and R′ are independently H, C1-C10 alkyl, or C6-C10 aryl.

In some embodiments, optional substituents include reactive groups such as groups crosslinkable by (4+2) cycloaddition, for example,

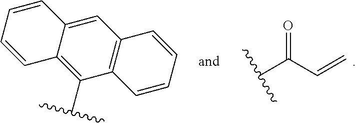

In some embodiments of Formula A, one or more of Z¹, Z², Q, X, and A can be optionally substituted with one or more reactive groups such as groups crosslinkable by (4+2) cycloaddition described above.

In some embodiments of Formula A, the compound is represented by Formula A1:

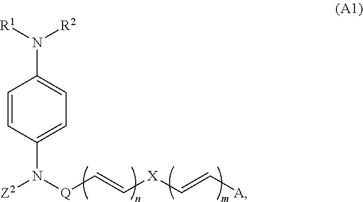

wherein:

Z², Q, X, A, n, and m are as defined above for Compound of Formula A; and

R¹ and R² are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

In some embodiments, the compound is represented by Formula A2:

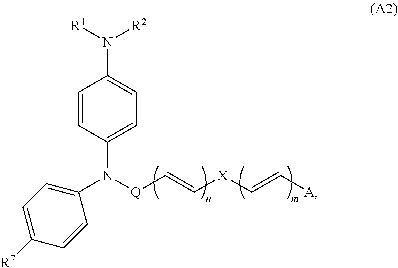

wherein:

Q, X, A, n, and m are as defined above;

R¹ and R² are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl;

R⁷ is H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl, optionally substituted C₁-C₁₀ alkyloxy, optionally substituted C₃-C₁₀ heteroalkyloxy, or NR³R⁴; and

R³ and R⁴ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

In some embodiments, the compound is represented by Formula A3:

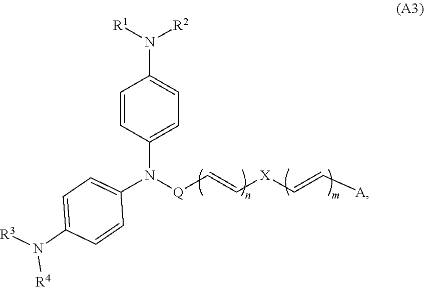

wherein:

Q, X, A, n, and m are as defined above;

R¹ and R² are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl; and

R³ and R⁴ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

In some embodiments, the compound is represented by Formula A4:

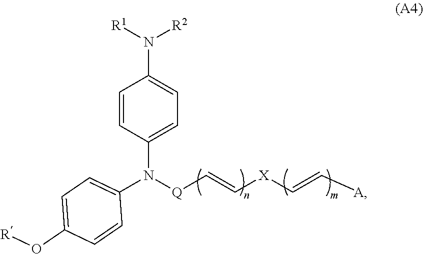

wherein:

Q, X, A, n, and m are as defined above for Compound A;

R¹ and R² are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl; and

R′is optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

In some embodiments of Formulae A1-A4, Q is:

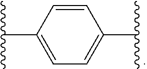 [SMILES: *c1ccc(*)cc1]

In some embodiments of Formulae A1-A4, Q is:

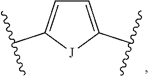

wherein J is i S, O, or NR⁸; and R⁸ is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl.

In some embodiments of Formulae A1-A4, Q is:

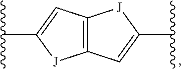

wherein J, at each occasion, is independently S, O, or NR⁸; and R⁸ is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl.

In some embodiments, J is S. In some embodiments, J is O. In some embodiments, J is NR⁸.

In some embodiments of Formulae A1-A4, A is:

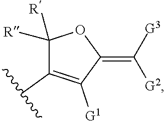

wherein R′ and R″ are independently selected from optionally substituted C1-C12 alkyl (e.g., fluorinated alkyl) and optionally substituted C6-C10 aryl (e.g., fluorinated aryl), and G¹, G², and G³ are independently selected from electronegative groups that include F, CN, CF₃, SO₂CF₃.

In some embodiments, R′ is CF₃. In other embodiments, R″ is phenyl. In certain embodiments, G¹, G², and G³ are CN.

In some embodiments, A is

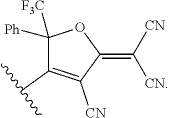 [SMILES: *C1=C(C#N)C(=C(C#N)C#N)OC1(c1ccccc1)C(F)(F)F]

In some embodiments, m is 1. In some embodiments, n is 1.

In some embodiments, the compound is a compound of Formula A5:

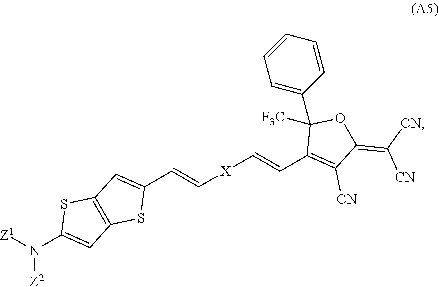

wherein:

Z¹ and Z² are independently H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl;

X is:

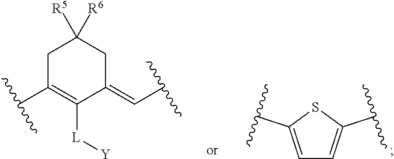

L is absent or L is S or O;

Y is H, optionally substituted C₁-C₂₀ alkyl, optionally substituted C₃-C₅₀ heteroalkyl, optionally substituted C₃-C₁₀ cycloalkyl, or optionally substituted C₃-C₁₀ cycloheteroalkyl; and

R⁵ and R⁶ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

In some embodiments, the compound is a compound of Formula A6:

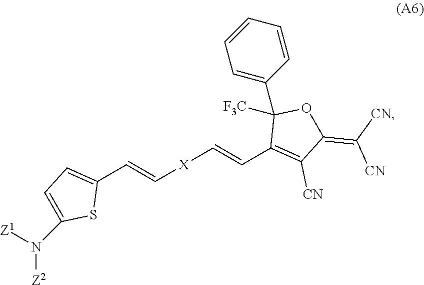

wherein:

Z¹ is optionally substituted C₆-C₁₀ aryl or optionally substituted C₅-C₁₀ heteroaryl;

Z² is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl;

X is:

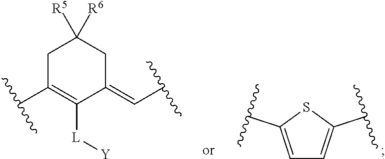

L is absent or L is S or O;

Y is H, optionally substituted C₁-C₂₀ alkyl, optionally substituted C₃-C₅₀ heteroalkyl, optionally substituted C₃-C₁₀ cycloalkyl, or optionally substituted C₃-C₁₀ cycloheteroalkyl; and

R⁵ and R⁶ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

In some embodiments, the compound is a compound of Formula A7:

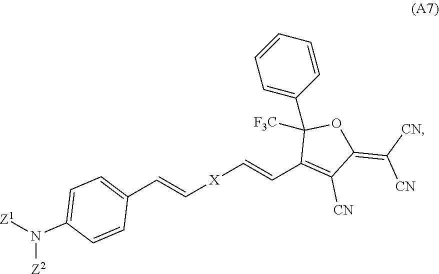

wherein:

Z¹ is optionally substituted C₆-C₁₀ aryl or optionally substituted C₅-C₁₀ heteroaryl;

Z² is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl;

X is:

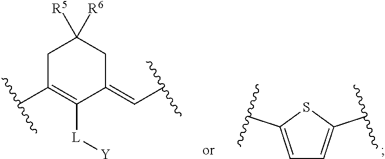

L is absent or L is S or O;

Y is H, optionally substituted C₁-C₂₀ alkyl, optionally substituted C₃-C₅₀ heteroalkyl, optionally substituted C₃-C₁₀ cycloalkyl, or optionally substituted C₃-C₁₀ cycloheteroalkyl; and

R⁵ and R⁶ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

The polarizable chromophores disclosed herein can comprise one or more reactive groups that can form a covalent bond (i.e., crosslink) when reacted with a counterpart group, for example, when subjected to high temperatures. Any suitable reactive groups and counterpart groups can be used to form the films comprising chromophores of the disclosure. In some embodiments, the reactive groups and counterpart groups are groups crosslinkable by (4+2) cycloaddition. A number of such groups is known in the art.

In some embodiments, one or more of Y, X, R¹, R², R³, R⁴, R⁵, and R⁶ comprise a reactive group. In some embodiments, one or more of Y, X, R¹, R², R³, R⁴, R⁵, and R⁶ are optionally substituted with one or more reactive group, for example, a group crosslinkable by (4+2) cycloaddition such as an anthracenyl group or an acrylate group. In some embodiments, the group crosslinkable by (4+2) cycloaddition is represented by the structure:

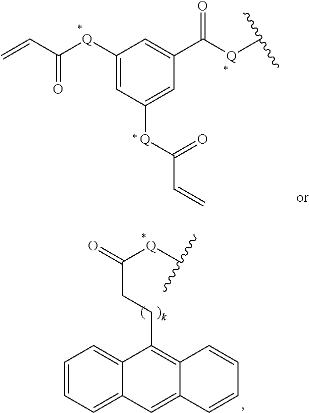

wherein k is 0, 1, 2, 3, 4, or 5, and each Q* is independently NH, N(C1-C10-alkyl), O, or S.

In some embodiments, one or more of Y, X, R¹, R², R³, R⁴, R⁵, and R⁶ are optionally substituted with one or more functional groups or protected functional groups, which can be present in addition to the one or more reactive groups described above. As used herein, a “functional group” is a group, a substituent, or moiety responsible for the characteristic chemical reactions of the molecule that comprises such functional group. For example, a hydroxyl functional group is a group that can undergo esterification reaction; a hydroxyl functional group can be protected with a silyl protective group, such as trimethylsilyl or tert-butyldiphenylsilyl (TBDPS).

In some embodiments of the Formulae of the disclosure, L-Y is H, OL¹OSiR¹⁰R¹¹R¹², or SL¹OSiR¹⁰R¹¹R¹², wherein L¹ is an optionally substituted C₂-C₂₀ alkylene or optionally substituted C₃-C₅₀ heteroalkylene; and R¹⁰, R¹¹, and R¹² are independently H, C1-C10 alkyl, or C6-C10 aryl. In some embodiments, L-Y is H. In some embodiments, L-Y is —SCH₂CH₂OH or SCH₂CH₂OTBDPS.

In some embodiments, R¹ is methyl or optionally substituted ethyl. In some embodiments, R² is methyl or optionally substituted ethyl. In some embodiments, R³ is methyl or optionally substituted ethyl. In some embodiments, R⁴ is methyl or optionally substituted ethyl.

In some embodiments, R⁵ is CH₃ and R⁶ is CH₃.

In some embodiments of the compounds disclosed herein, at least one of Z¹ and X is substituted with a group selected from

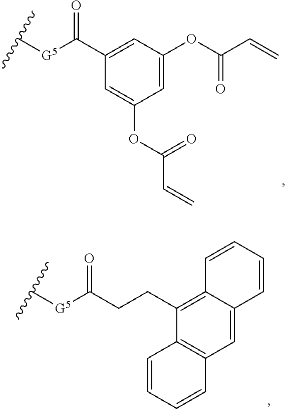

and OSiR¹⁰R¹¹R¹²,

wherein G⁵ is NH, O, S, or N(C1-C10-alkyl) and R¹⁰, R¹¹, and R¹² are independently H, optionally substituted C1-C10 alkyl, or optionally substituted C6-C10 aryl.

In some embodiments of the compounds disclosed herein, L-Y is H, OL¹OSiR¹⁰R¹¹R¹², or SL¹OSiR¹⁰R¹¹R¹², wherein L¹ is an optionally substituted C2-C20alkylene or optionally substituted C3-C50 heteroalkylene; and R¹⁰, R¹¹, and R¹² are independently H, optionally substituted C1-C10 alkyl, or optionally substituted C6-C10 aryl.

In some embodiments of the compounds disclosed herein, X is:

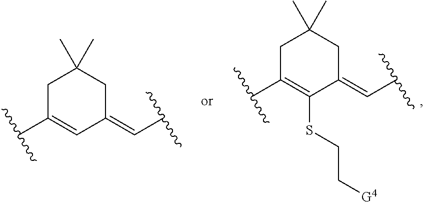

wherein G⁴ is OSiR¹⁰R¹¹R¹², wherein R¹⁰, R¹¹, and R¹² are independently H, optionally substituted C1-C10 alkyl, or optionally substituted C6-C10 aryl, or G⁴ is

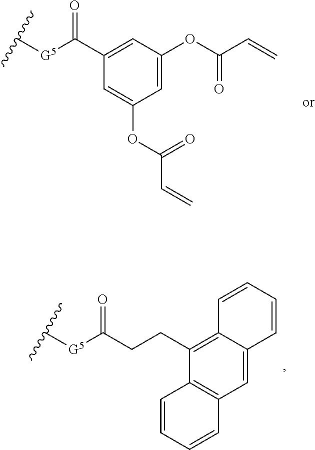

wherein G⁵ is NH, O, S, or N(C1-C10-alkyl).

In some embodiments, X is:

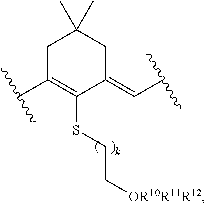

wherein k is an integer from 1 to 20 and R¹⁰, R¹¹, and R¹² are independently H, C1-C10 alkyl, or C6-C10 aryl.

In some embodiments, X is:

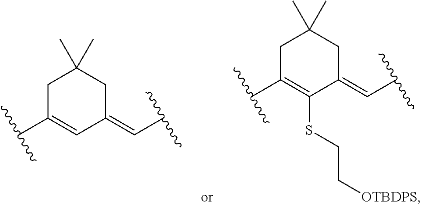

wherein TBDPS is

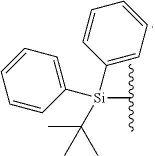 [SMILES: *[Si](c1ccccc1)(c1ccccc1)C(C)(C)C]

In some embodiments, the compound is a compound of Formula A8:

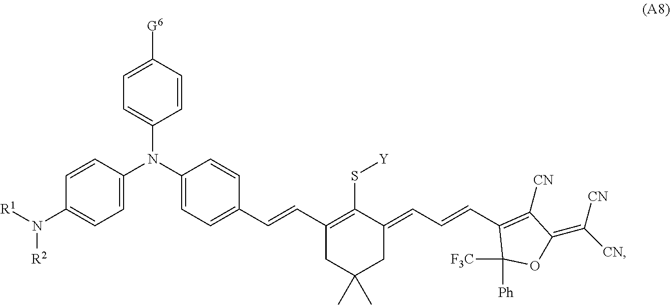

wherein:

G⁶ is OR′ or NR′R″, wherein R′ and R″ are independently optionally substituted C1-C10 alkyl;

R¹ is H or an optionally substituted C1-C10 alkyl,

R² is optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl; and

Y is optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl; and

wherein at least one of R², Y, and G⁶ is substituted with a group selected from

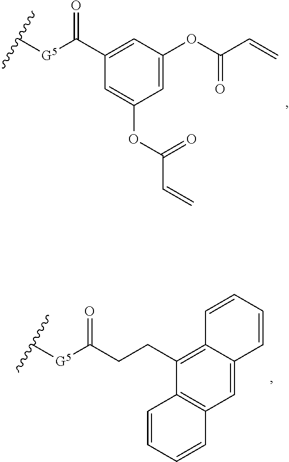

and OSiR¹⁰R¹¹R¹²,

wherein G⁵ is NH, O, S, or N(C1-C10-alkyl) and R¹⁰, R¹¹, and R¹² are independently H, optionally substituted C1-C10 alkyl, or optionally substituted C6-C10 aryl.

In some embodiments, the compound is Compound I, Compound II, Compound III, or Compound IV, Compound V, Compound VI, Compound VII, Compound VIII, Compound IX, Compound X, Compound XI, Compound XII, Compound XIII, Compound XIV, or Compound XV:

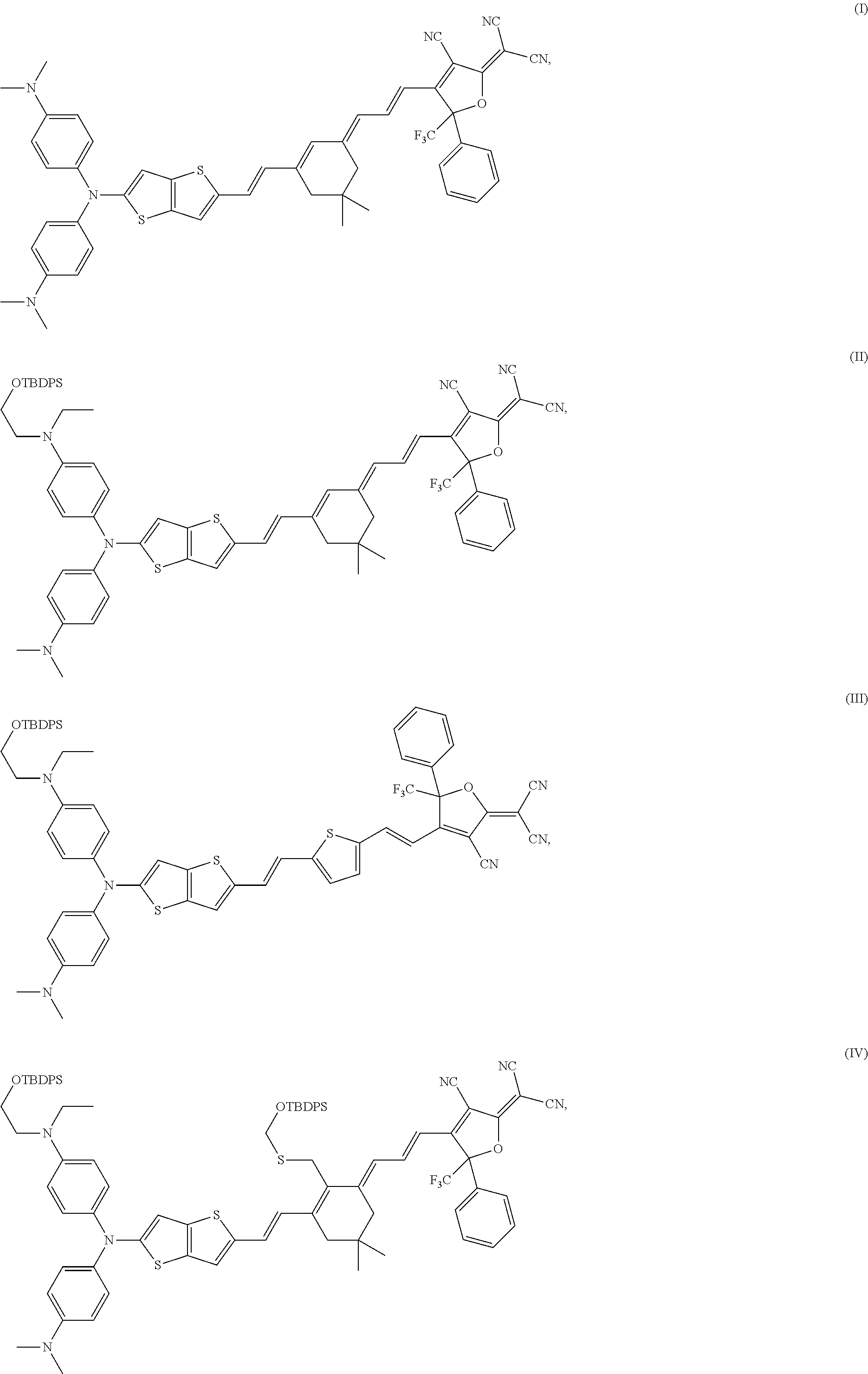

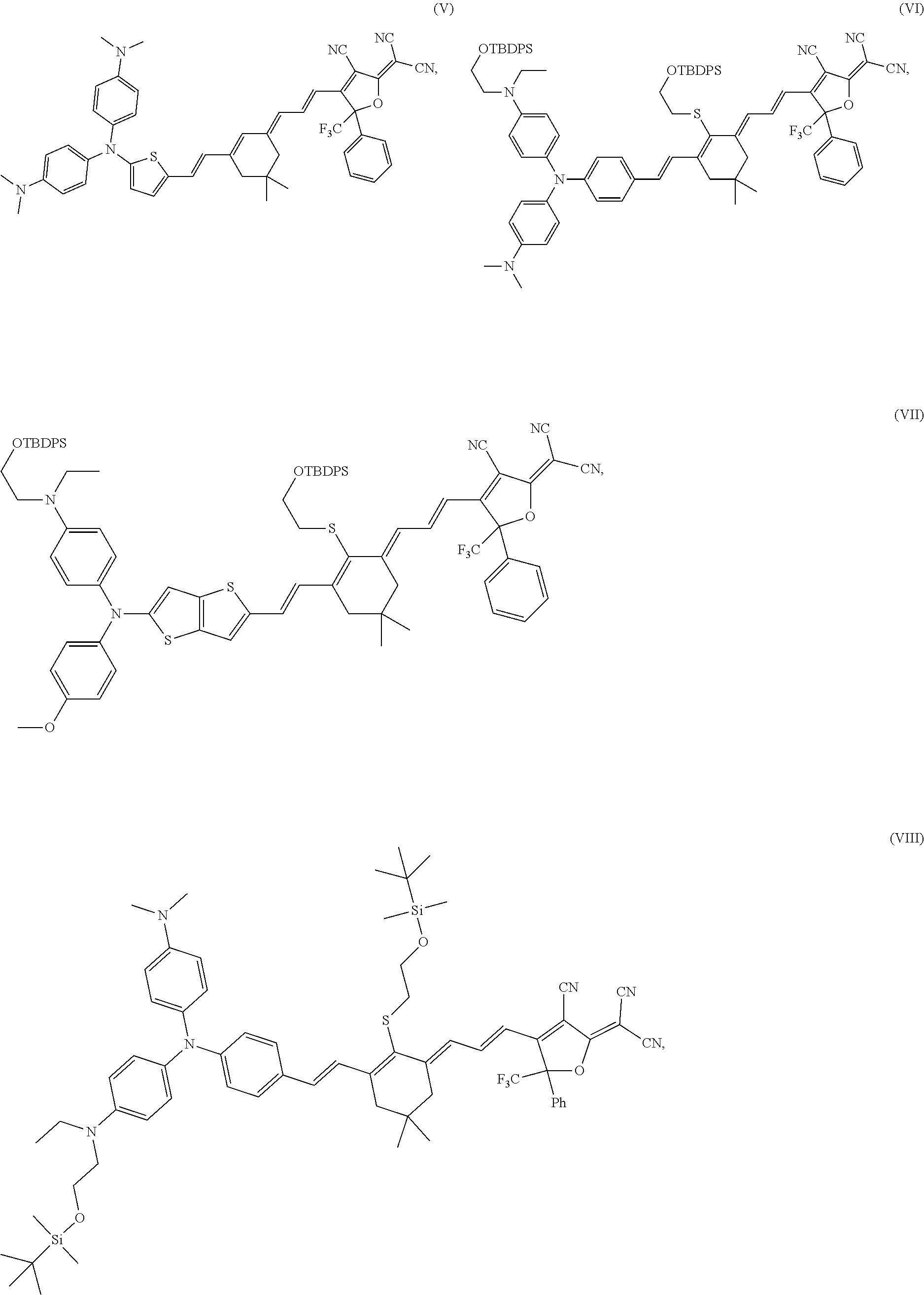

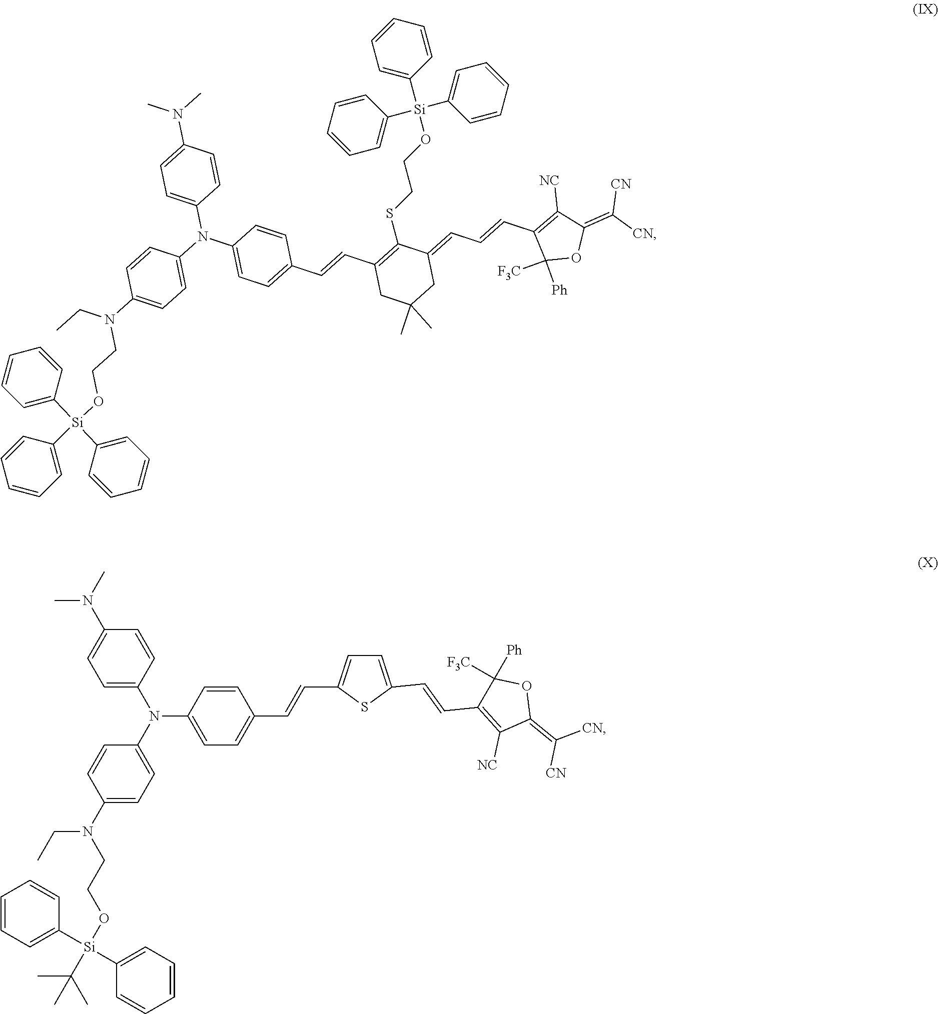 [SMILES: CCN(CCO[Si](c1ccccc1)(c1ccccc1)c1ccccc1)c1ccc(N(c2ccc(/C=C/C3=C(SCCO[Si](c4ccccc4)(c4ccccc4)c4ccccc4)C(=C/C=C/C4=C(C#N)C(=C(C#N)C#N)OC4(c4ccccc4)C(F)(F)F)/CC(C)(C)C3)cc2)c2ccc(N(C)C)cc2)cc1 ; CCN(CCO[Si](c1ccccc1)(c1ccccc1)C(C)(C)C)c1ccc(N(c2ccc(/C=C/c3ccc(/C=C/C4=C(C#N)C(=C(C#N)C#N)OC4(c4ccccc4)C(F)(F)F)s3)cc2)c2ccc(N(C)C)cc2)cc1]

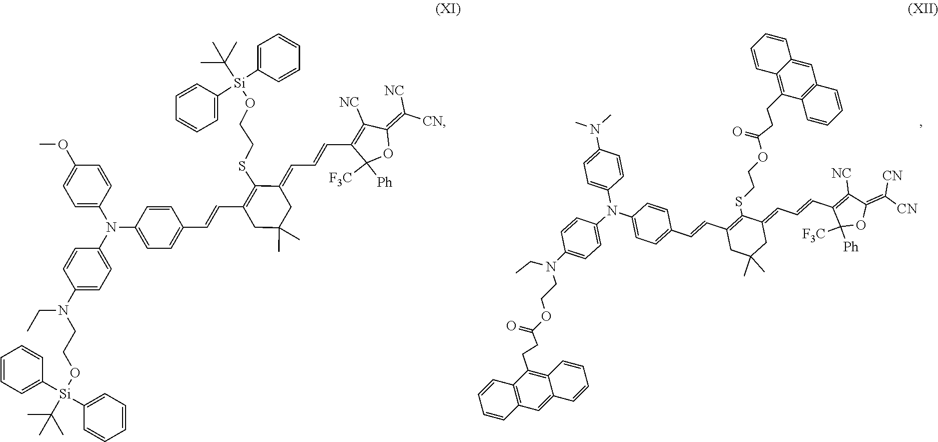 [SMILES: CCN(CCO[Si](c1ccccc1)(c1ccccc1)C(C)(C)C)c1ccc(N(c2ccc(/C=C/C3=C(SCCO[Si](c4ccccc4)(c4ccccc4)C(C)(C)C)C(=C/C=C/C4=C(C#N)C(=C(C#N)C#N)OC4(c4ccccc4)C(F)(F)F)/CC(C)(C)C3)cc2)c2ccc(OC)cc2)cc1 ; CCN(CCOC(=O)CCc1c2ccccc2cc2ccccc12)c1ccc(N(c2ccc(/C=C/C3=C(SCCOC(=O)CCc4c5ccccc5cc5ccccc45)C(=C/C=C/C4=C(C#N)C(=C(C#N)C#N)OC4(c4ccccc4)C(F)(F)F)/CC(C)(C)C3)cc2)c2ccc(N(C)C)cc2)cc1]

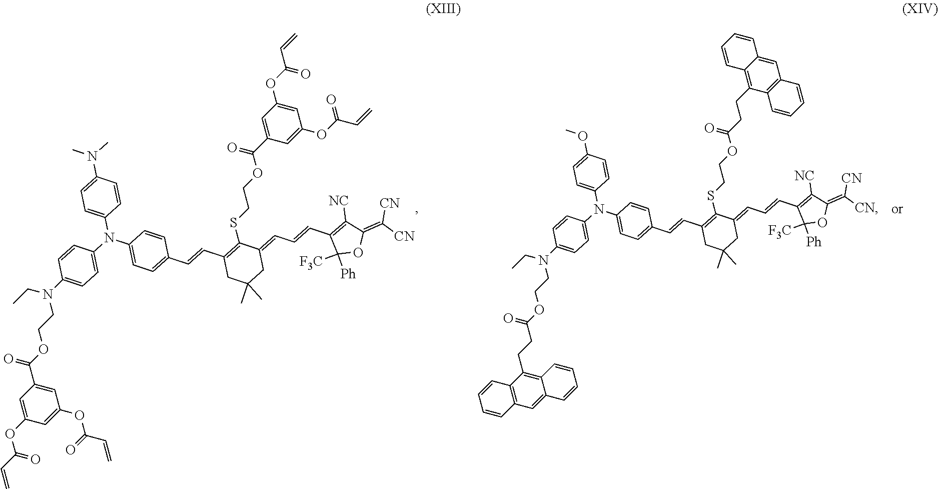 [SMILES: C=CC(=O)Oc1cc(OC(=O)C=C)cc(C(=O)OCCSC2=C(/C=C/c3ccc(N(c4ccc(N(C)C)cc4)c4ccc(N(CC)CCOC(=O)c5cc(OC(=O)C=C)cc(OC(=O)C=C)c5)cc4)cc3)CC(C)(C)C/C2=C\C=C\C2=C(C#N)C(=C(C#N)C#N)OC2(c2ccccc2)C(F)(F)F)c1 ; CCN(CCOC(=O)CCc1c2ccccc2cc2ccccc12)c1ccc(N(c2ccc(/C=C/C3=C(SCCOC(=O)CCc4c5ccccc5cc5ccccc45)C(=C/C=C/C4=C(C#N)C(=C(C#N)C#N)OC4(c4ccccc4)C(F)(F)F)/CC(C)(C)C3)cc2)c2ccc(OC)cc2)cc1]

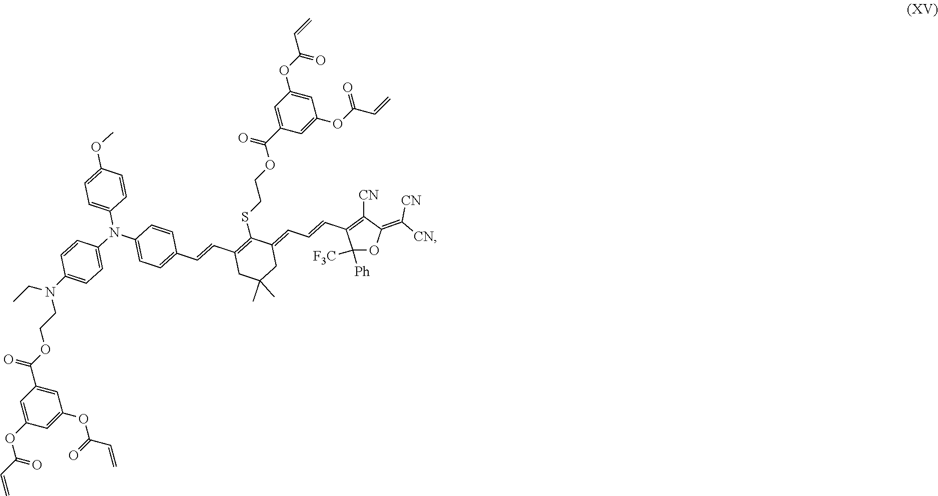 [SMILES: C=CC(=O)Oc1cc(OC(=O)C=C)cc(C(=O)OCCSC2=C(/C=C/c3ccc(N(c4ccc(OC)cc4)c4ccc(N(CC)CCOC(=O)c5cc(OC(=O)C=C)cc(OC(=O)C=C)c5)cc4)cc3)CC(C)(C)C/C2=C\C=C\C2=C(C#N)C(=C(C#N)C#N)OC2(c2ccccc2)C(F)(F)F)c1]

wherein TBDPS is

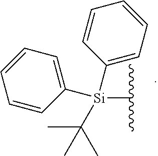 [SMILES: *[Si](c1ccccc1)(c1ccccc1)C(C)(C)C]

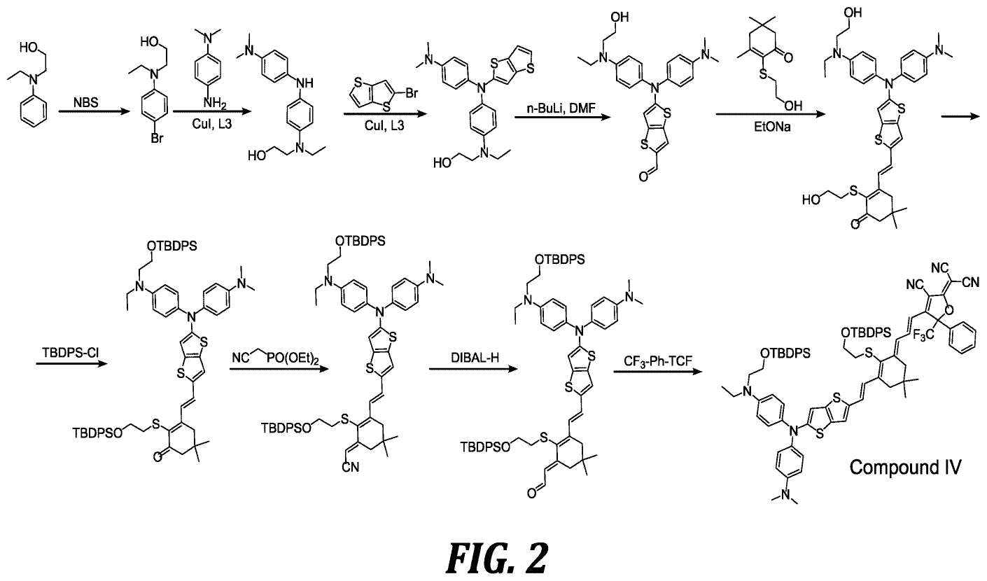

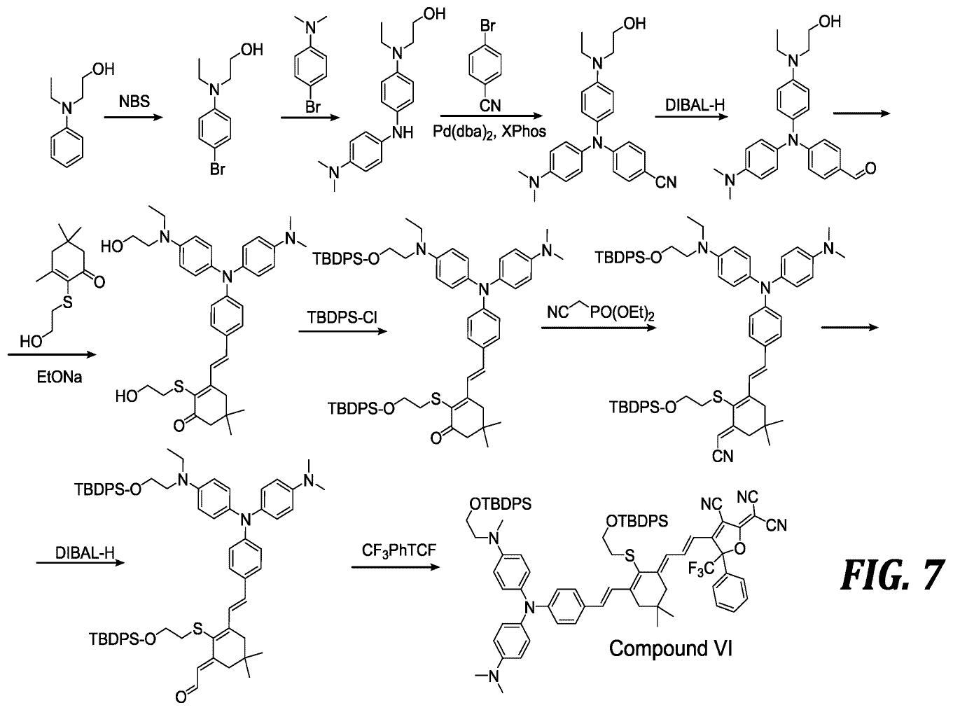

The chromophores disclosed herein, such as compounds of Formulae A, A1, A2, A3, A4, A5, A6, A7, A8, I, II, III, IV, V, VI, VII, VIII, IX, XI, XII, XIII, XIV, and XV can be made using standard organic synthetic methods known to those skilled in the art using the intermediates common to a number known OEO chromophores, including substituted thioether-substituted isophorones and the CF₃-phenyl substituted tricyanofuran (TCF) acceptor, such as those described in Dalton, L. R.; Sullivan, P. A.; Bale, D. H., Electric Field Poled Organic Electro-optic Materials: State of the Art and Future Prospects. Chemical Reviews 2010, 110 (1), 25-55, the disclosure of which is incorporated herein by reference in its entirety. Synthesis of exemplary chromophores is shown in FIG. 2 and FIG. 7.

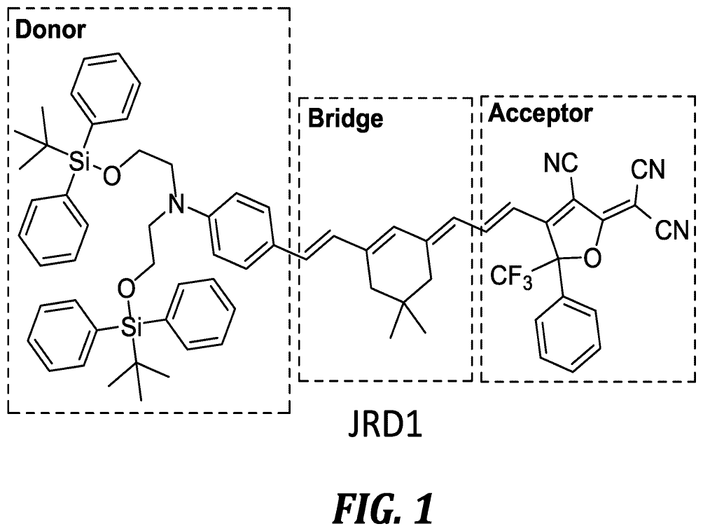

In some embodiments, the chromophores disclosed herein have a large static hyperpolarizability. In certain embodiments, the chromophores disclosed herein have a static hyperpolarizability between about one and a half times to about three times that of a reference chromophore JRD1 (FIG. 1), for example, as measured by Hyper-Rayleigh Scattering at 1300 nm in chloroform solution and extrapolated to zero frequency using a damped two-level model. In some embodiments, the chromophores disclosed herein have a static hyperpolarizability in excess of between about one and a half times to about three times that of a reference chromophore JRD1.

In another aspect, provided herein are polymeric compositions comprising the chromophores disclosed herein. In certain embodiments, the chromophores are blended with a polymer to form a processible and durable film that can have electro-optic activity induced by electric field poling, a process in which a strong, direct-current electric field is applied to the film, the film is heated to near its glass transition temperature (T_{g}) to enable chromophores to re-orient such that their dipole moments have net alignment with the field, and then cooled while still in the presence of the field to retain the poling-induced order. Electro-optic activity can be measured by Teng-Man ellipsometry, attenuated total reflection (ATR), methods known to those skilled in the art.

In certain embodiments, films containing the chromophores disclosed herein blended with polymethylmethyacrylate (PMMA) have a r₃₃ value from about 70 pm/V to about 300 pm/V, from about 40 pm/V to about 140 pm/V, from about 30 pm/V to about 250 pm/V, from about 75 pm/V to about 300 pm/V, from about 80 pm/V to about 250 pm/V, from about 50 pm/V to 200 pm/V, from about 15 to about 105 pm/V, from about 105 to about 405 pm/V, from about 125 to about 1100 pm/V, from about 125 to about 1200 pm/V, from about 125 to about 1300 pm/V, from about 125 to about 1500 pm/V, in excess of about 350 pm/V, in excess of about 500 pm/V, in excess of about 750 pm/V, or in excess of about 1000 pm/V, as measured by Teng-Man ellipsometry.

In some embodiments, the films comprising the chromophores described herein can be combined with films of a dielectric material or wide-bandgap semiconductor as a charge blocking layer, for example, in order to minimize conductivity during poling of the film. Such films can comprise organic materials such as poly (benzocyclobutene) (BCB, Cyclotene™) or inorganic materials, including but not limited to, TiO₂, MoO₃, ZrO₂, HfO₂, SiO₂, Al₂O₃, Si₃N₄, or combinations thereof. In some embodiments, the films comprise poly (benzocyclobutene). In some embodiments, the BCB layer has a thickness from about 40 nm to about 150 nm or from about 60 nm to about 100 nm. In some embodiments, the charge blocking layer are such as the layers described in “Benzocyclobutene barrier layer for suppressing conductance in nonlinear optical devices during electric field poling” Applied Physics Letters 104, 243304 (2014).

In yet another aspect, provided herein are electro-optic devices comprising the films disclosed herein or films formed by the methods disclosed herein. Exemplary devices incorporating the films of the disclosure include an electro-optic modulator, antenna, Mach-Zehnder modulator, phase modulator, silicon-organic hybrid modulator, plasmonic-organic hybrid modulator, electrical-to-optical convertor, terahertz detector, frequency shifter, or frequency comb source. In some embodiments, the electro-optic devices comprising the films disclosed herein further comprise one or more charge blocking layers as described above.

Certain components of optical communications systems can be fabricated, in whole or part, with the films according to the present disclosure. Exemplary components include, without limitation, straight waveguides, bends, single-mode splitters, couplers (including directional couplers, MMI couplers, star couplers), routers, filters (including wavelength filters), switches, modulators (optical and electro-optical, e.g., birefringent modulator, the Mach-Zehnder interferometer, and directional and evanescent coupler), arrays (including long, high-density waveguide arrays), optical interconnects, optochips, single-mode DWDM components, photonic crystal devices, resonant devices (e.g., photonic crystal, ring, or disc resonators, and gratings. The films described herein may be used with, for example, wafer-level processing, as applied in, for example, vertical cavity surface emitting laser (VCSEL) and CMOS technologies.

In many applications, the films described herein can be used in lieu of lithium niobate, gallium arsenide, and other inorganic materials that currently find use as light-transmissive materials in optical communication systems.

Unless specifically defined herein, all terms used herein have the same meaning as they would to one skilled in the art of the present invention.

As used herein, the term “about” indicates that the subject value can be modified by plus or minus 5% and still fall within the disclosed embodiment.

The use of the term “or” in the claims is used to mean “and/or” unless explicitly indicated to refer to alternatives only or the alternatives are mutually exclusive, although the disclosure supports a definition that refers to only alternatives and “and/or.” The words “a” and “an,” when used in conjunction with the word “comprising” in the claims or specification, denote one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A) B” means (B) or (AB) that is, A is an optional element. Additionally, the words “herein,” “above,” and “below,” and words of similar import, when used in this application, shall refer to this application as a whole and not to any particular portions of the application.

All publications cited herein and the subject matter for which they are cited are hereby specifically incorporated by reference in their entireties.

The following examples are provided to illustrate certain particular features and/or embodiments of the disclosure. The examples should not be construed to limit the disclosure to the particular features or embodiments described.

### EXAMPLES

Using density functional theory (DFT) calculations performed using well-validated methods, the inventors found that the chromophores of the disclosure, such as Compounds I and II, can have large hyperpolarizability. Calculations were performed at the

M062X/6-31+G(d) level of theory in a chloroform implicit solvent environment (PCM), with hyperpolarizabilities calculated using analytic differentiation (CPHF). Identical calculations were also performed on a truncated model of JRD1 as a standard; the OTBDPS groups on the donor in JRD1 were replaced with hydrogens for computational efficiency, modeling the donor as a diethylaniline. Computed hyperpolarizabities were reported in reference to this model. Exemplary Compounds I-XV were synthesized using standard organic synthesis methods known to those skilled in the art, and composition was verified by proton (¹H) NMR and electrospray ionization mass spectrometry (ESI-MS). UV/Visible absorbance spectra were measured in both chloroform and thin films using methods known to those skilled in the art.

Hyperpolarizabilities of exemplary Compounds I, II, III, IV, V, VI, and VII in chloroform solution were determined by hyper-Rayleigh scattering (HRS) with light produced by a high-repetition rate (80 MHz) broadband (680-1300 nm) femtosecond-pulsed laser (Insight DS+, Spectra-Physics) tuned to 1300 nm, as previously described (Campo, J.; Desmet, F.; Wenseleers, W.; Goovaerts, E., Highly sensitive setup for tunable wavelength hyper-Rayleigh scattering with parallel detection and calibration data for various solvents. Optics Express 2009, 17 (6), the disclosure of which is incorporated herein by reference). Measurements were performed relative to a pure chloroform standard, and data was extrapolated to zero frequency using the damped two-level model and a linewidth of 0.1 eV. In order to provide a clearer comparison to the state of the art and remove dependence on the reference value used for chloroform, state-of-the-art chromophore JRD1 was measured in the same experiment set and data is also reported relative to JRD1. Data from DFT, UV/Vis, and HRS measurements are summarized in Table 1.

TABLE 1
Key optical properties for exemplary chromophores

| | λₘₐₓ | λₘₐₓ | β₀/ | β₀/ |
|---|---|---|---|---|
| Com- | in CHCl₃ | in neat | β_{0,JRD1} | β_{0,JRD1} |
| pound | (nm) | film (nm) | (DFT) | (HRS) |
| JRD1 | 784 | 900 | 1 (ref) | 1 (ref) |
| I | 1058 | 1105 | 2.16 | 2.85 ± 0.10 |
| II | 1068 | 1103 | 2.19 | 2.60 ± 0.04 |
| III | 943 | 951 | 1.85 | 2.47 ± 0.04 |
| IV | 1081 | 1114 | 2.32 | 3.34 ± 0.04 |
| V | 1028 | — | 1.76 | 1.18 ± 0.12 |
| VI | 897 | 935 | 1.52 | 2.15 ± 0.09 |
| VII | 1020 | 1097 | 1.76 | 2.83 ± 0.17 |

Exemplary thin films comprising exemplary Compounds I, II, III, IV, VI, and VII were formed on glass slides and/or glass slides on which half of the slide was coated with a conductive layer of indium tin oxide (ITO); ITO slides were fabricated by Thin Film Devices (Anaheim, CA). Chromophores were deposited by spin coating from trichloroethane (TCE) solutions containing the chromophore, either neat or blended with PMMA. Films were dried in a vacuum oven at 65° C. before use.

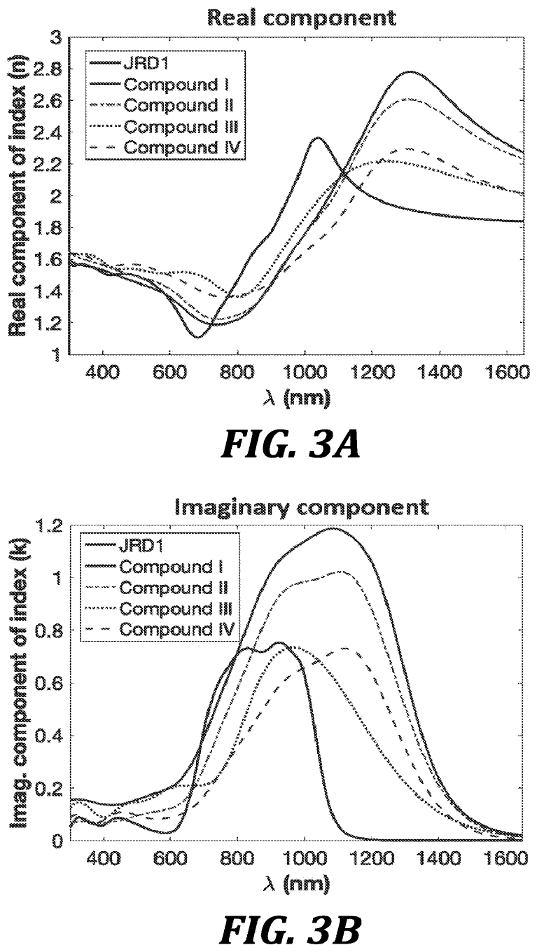

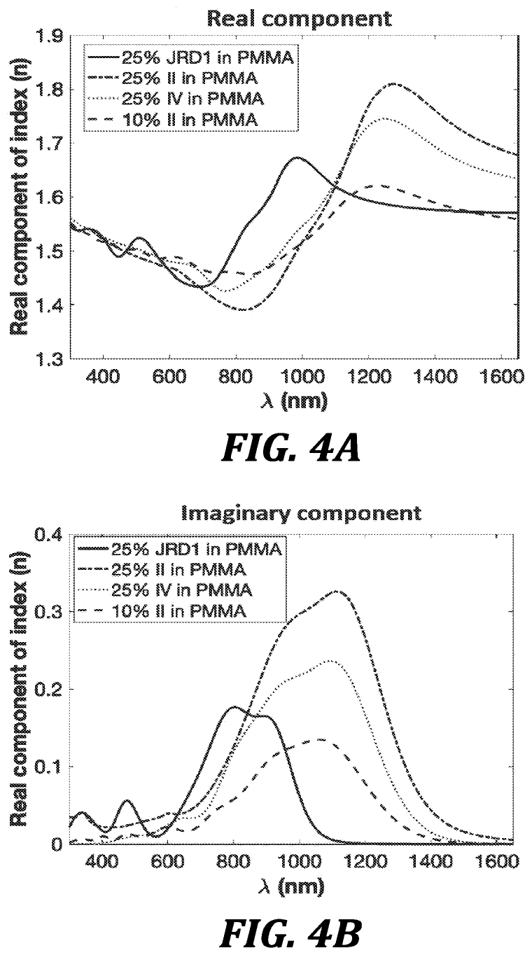

The complex refractive indices (n and k) of thin films comprising exemplary Compounds I, II, III, and IV on glass substrates were determined using Variable Angle Spectroscopic Ellipsometry (VASE) at the UW Molecular Analysis Facility using a JA Woollam MC2000 spectroscopic ellipsometer and previously reported methods (Benight, S. J.; Johnson, L. E.; Barnes, R.; Olbricht, B. C.; Bale, D. H.; Reid, P. J.; Eichinger, B. E.; Dalton, L. R.; Sullivan, P. A.; Robinson, B. H., Reduced Dimensionality in Organic Electro-Optic Materials: Theory and Defined Order. The Journal of Physical Chemistry B 2010, 114 (37), 11949-11956, the disclosure of which is incorporated herein by reference). Data for exemplary Compounds I, II, III, and IV as neat films is shown in FIG. 3. Data for films of Compound II in PMMA and compared with neat JRD1 is shown in FIG. 4. The data in FIG. 4 is representative for the behavior of the compounds disclosed herein when blended in dilute polymer films.

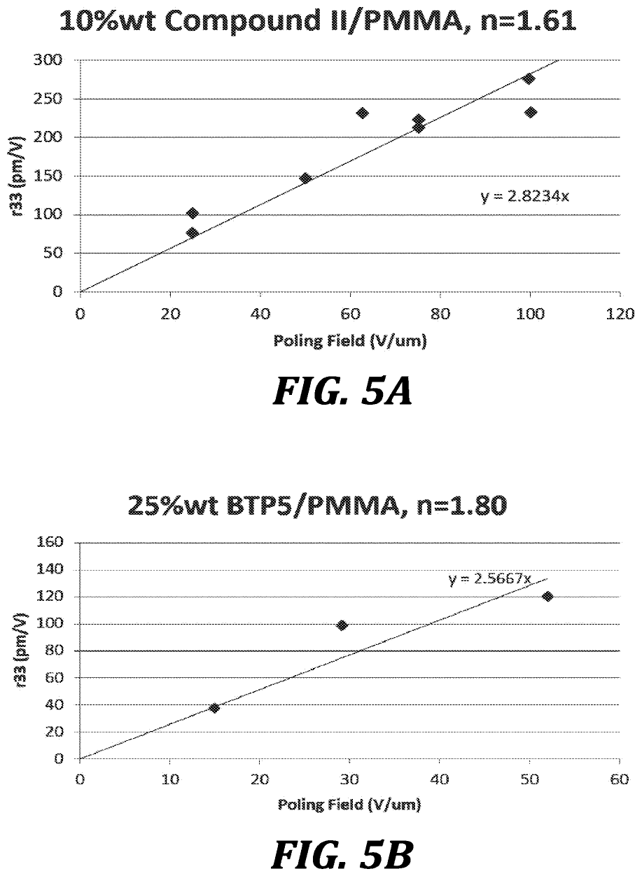

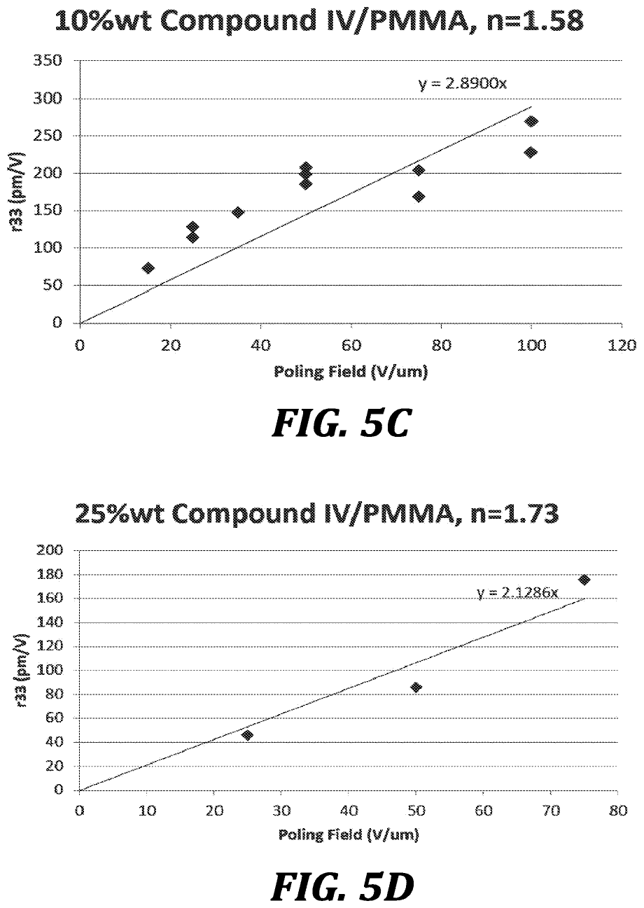

Films for electro-optic measurements were prepared by sputter-coating gold electrodes on thin films of exemplary Compounds I, II, III, IV, VI, and VII attaching wires using commercially available silver paste, poling at a temperature appropriate for the T_{g} of the material (approximately 115° C. for blends in PMMA, 85-90° C. for neat materials) using a poling field of Eₚ with a strength in the 15 V/μm to 110 V/μm range, cooling to <35° C. and measuring electro-optic activity at 1310 nm using previously reported methods and instrumentation (Dalton, L. R.; Sullivan, P. A.; Bale, D. H., Electric Field Poled Organic Electro-optic Materials: State of the Art and Future Prospects. Chemical Reviews 2010, 110 (1), 25-55, the disclosure of which is incorporated herein by reference). Representative poling results for exemplary Compounds II and III are shown in FIG. 5. Poling efficiencies r₃₃/Eₚ were determined by linear fitting of the electro-optic activity (in pm/V) versus Eₚ.

In addition to the data shown in FIG. 5, exemplary Compound I at 10% concentration by mass in PMMA demonstrated a poling efficiency of 1.77 nm²/V² and Compound III at 25% concentration by mass in PMMA demonstrated a poling efficiency of 2.87 nm²/V². The poling efficiencies compare highly favorably to 25% JRD1 in PMMA (˜1 nm²/V²). The poling efficiencies of exemplary Compounds II and IV at low concentrations are particularly extraordinary for their number density. Exemplary Compound II at 10% concentration in PMMA (2.82 nm²/V²) and exemplary Compound IV at 10% concentration in PMMA (2.86 nm²/V²) were competitive with neat JRD1, which has a poling efficiency of 3.43±0.2 nm²/V² with a BCB charge blocking layer between the ITO and OEO material and 3.1±0.1 without a charge blocking layer, as disclosed in Jin, W.; Johnston, P. V.; Elder, D. L.; Tillack, A. F.; Olbricht, B. C.; Song, J.; Reid, P. J.; Xu, R.; Robinson, B. H.; Dalton, L. R., Benzocyclobutene barrier layer for suppressing conductance in nonlinear optical devices during electric field poling. Applied Physics Letters 2014, 104 (24), 243304, the disclosure of which is incorporated herein by reference). The 2.5×+ improvement in EO activity at a given concentration in a polymer host demonstrates the utility of the exemplary chromophores comprising the novel thienothiophene-derived donor such as exemplary Compounds I, II, III, and IV for electro-optic devices, enabling high device performance while providing substantial flexibility for blending with chromophores.

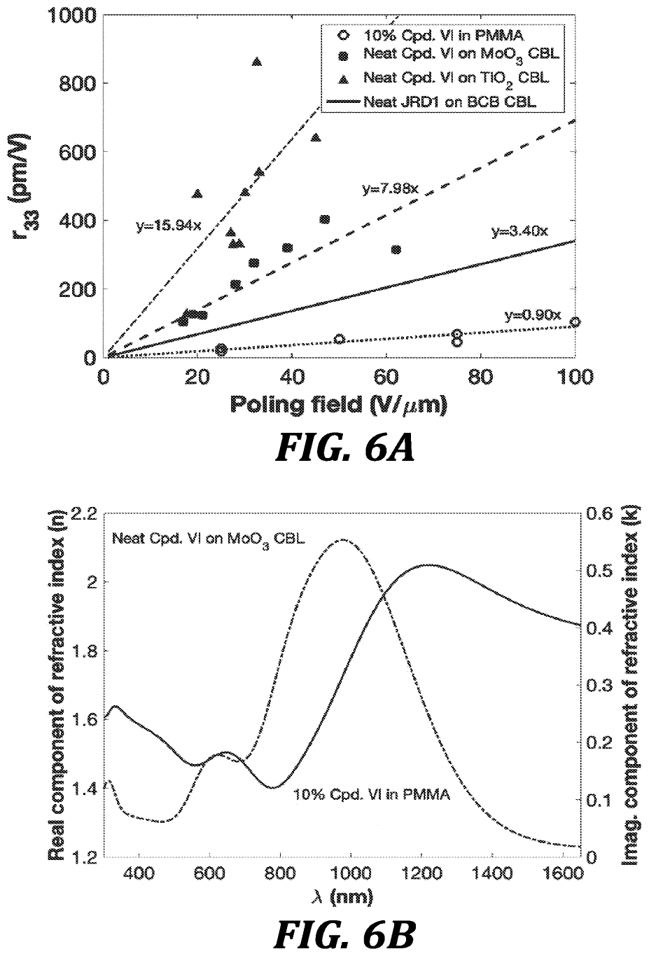

Exemplary Compounds V, VI, and VII showed a range of structural variants of the invention utilizing different aromatic groups and side-chain substitutions in the electron-donating region of the molecule, which allow desired substitutions and tuning of optical properties while demonstrating an improvement in hyperpolarizability over state-of-the-art compound JRD1, as shown in Table 1. Exemplary Compound VI shows competitive poling efficiency at 10% concentration in PMMA (0.90 nm²/V²) to 25% JRD1 in PMMA, and exceptional poling efficiencies as a neat material (100% concentration, no polymer) when combined with a charge blocking layer to reduce current during poling. A poling efficiency of 7.98 was obtained in combination with a MoO₃ (20 nm thick, deposited via evaporation) charge blocking layer; a poling efficiency of 15.98 was obtained with a sol-gel TiO₂ charge blocking layer. The latter has led to r₃₃ values in excess of 600 pm/V, substantially exceeding the record value for JRD1 and exceeding the poling efficiency of JRD1 by nearly a factor of 5. The real component of the refractive index of exemplary Compound VI is also enhanced, reaching values of 2.02 at 1310 nm and 1.90 at 1550 nm, enabling exceptional n³r₃₃ values. Refractive index and poling data for Compound VI is shown in FIG. 6. Exemplary Compound VII demonstrated a poling efficiency of 0.94 nm²/V² at 10% concentration in PMMA, slightly exceeding that of 10% exemplary Compound VI in PMMA.

While illustrative embodiments have been illustrated and described, it will be appreciated that various changes can be made therein without departing from the spirit and scope of the invention.

1. A compound of Formula A2: 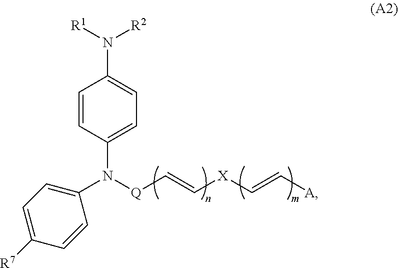
wherein:
A is a π-electron acceptor group;
X is: 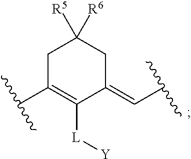
L is absent or L is S or O;
Y is H, optionally substituted C₁-C₂₀ alkyl, optionally substituted C₃-C₅₀ heteroalkyl, optionally substituted C₃-C₁₀ cycloalkyl, optionally substituted C₃-C₁₀ cycloheteroalkyl;
n is 1, 2, or 3;
m is 1, 2, or 3, and
R⁵ and R⁶ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl;
Q is 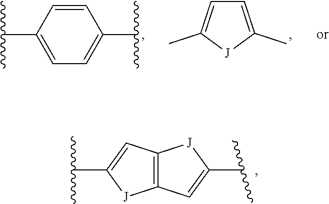
J, at each occasion, is independently S, O, or NR⁸;
R⁸ is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl; and
R¹ and R² are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl;
R⁷ is H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl, optionally substituted C₁-C₁₀ alkyloxy, optionally substituted C₃-C₁₀ heteroalkyloxy, or NR³R⁴; and
R³ and R⁴ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

2. The compound of claim 1, wherein the compound is represented by Formula A3: 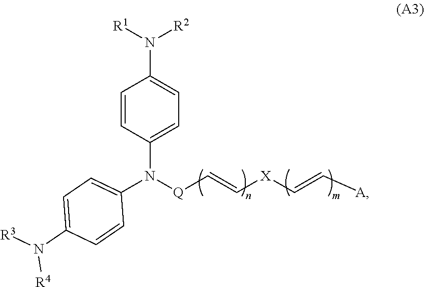
wherein:
R¹ and R² are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl; and
R³ and R⁴ are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

3. The compound of claim 1, wherein the compound is represented by Formula A4: 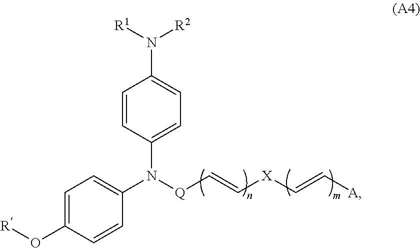
wherein:
R¹ and R² are independently H, optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl; and
R′is optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl.

4. The compound of claim 1, wherein Q is:

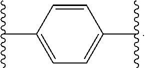 [SMILES: *c1ccc(*)cc1]

5. The compound of claim 1, wherein O is: 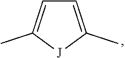
wherein:
J is S, O, or NR⁸; and
R⁸ is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl.

6. The compound of claim 1, wherein Q is: 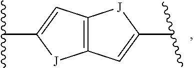
wherein:
J, at each occasion, is independently S, O, or NR⁸; and
R⁸ is H, optionally substituted C₁-C₁₀ alkyl, optionally substituted C₃-C₁₀ heteroalkyl, optionally substituted C₃-C₁₀ cycloalkyl, optionally substituted C₃-C₁₀ cycloheteroalkyl, optionally substituted C₆-C₁₀ aryl, or optionally substituted C₅-C₁₀ heteroaryl.

7. The compound of claim 5, wherein J is S.

8. The compound of claim 1, wherein A is 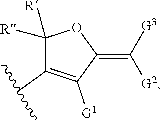
wherein R′ and R″ are independently selected from optionally substituted C1-C12 alkyl and optionally substituted C6-C10 aryl, and G¹, G², and G³ are independently selected from F, CN, CF₃, SO₂CF₃.

9. The compound of claim 1, wherein A is

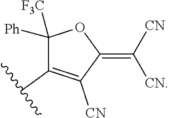 [SMILES: *C1=C(C#N)C(=C(C#N)C#N)OC1(c1ccccc1)C(F)(F)F]

10. The compound of claim 1, wherein m is 1.

11. The compound of claim 1, wherein n is 1.

12. The compound of claim 1, wherein the compound is a compound of 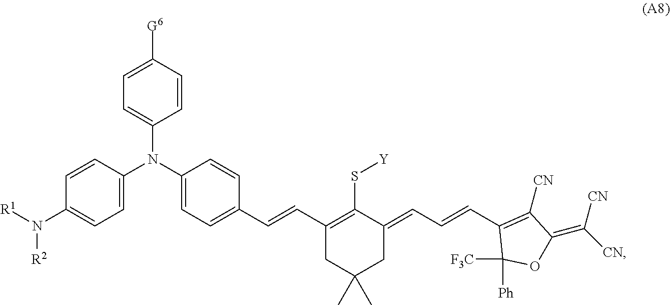
wherein:
G⁶ is OR′ or NR′R″, wherein R′ and R″ are independently optionally substituted C1-C10 alkyl;
R¹ is H or an optionally substituted C1-C10 alkyl,
R² is optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl; and
Y is optionally substituted C₁-C₁₀ alkyl, optionally substituted C₃-C₁₀ heteroalkyl, optionally substituted C₃-C₁₀ cycloalkyl, or optionally substituted C₃-C₁₀ cycloheteroalkyl; and
wherein at least one of R², Y, and G⁶ is substituted with a group selected from 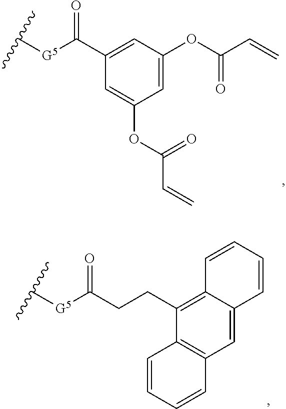
and OSiR¹⁰R¹¹R¹²,
wherein G⁵ is NH, O, S, or N(C1-C10-alkyl) and R¹⁰, R¹¹, and R¹² are independently H, optionally substituted C1-C10 alkyl, or optionally substituted C6-C10 aryl.

13. The compound of claim 1, wherein the compound is Compound I, Compound II, Compound IV, Compound V, Compound VI, Compound VII, Compound VIII, Compound IX, Compound XI, Compound XII, Compound XIII, Compound XIV, or Compound XV: 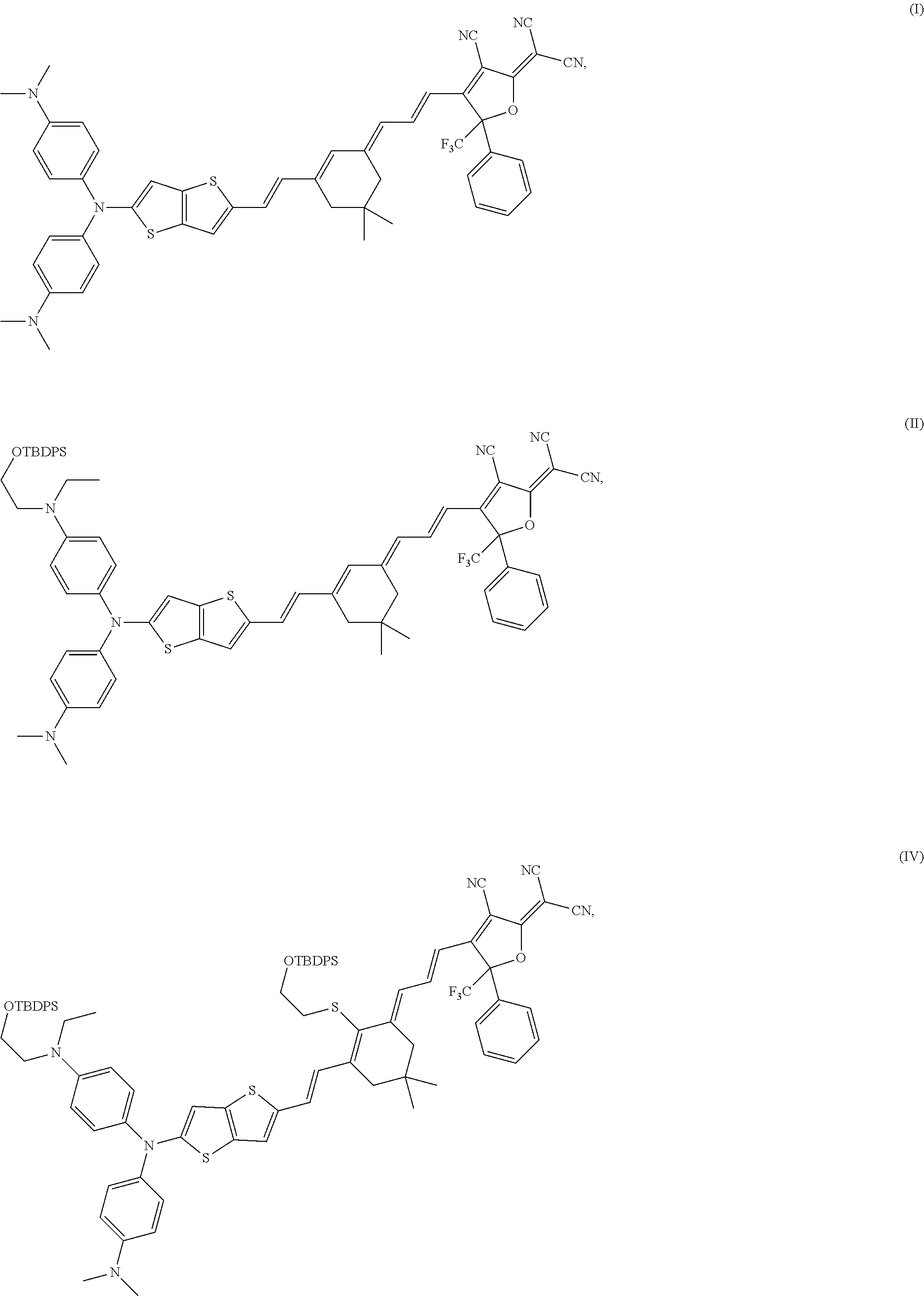 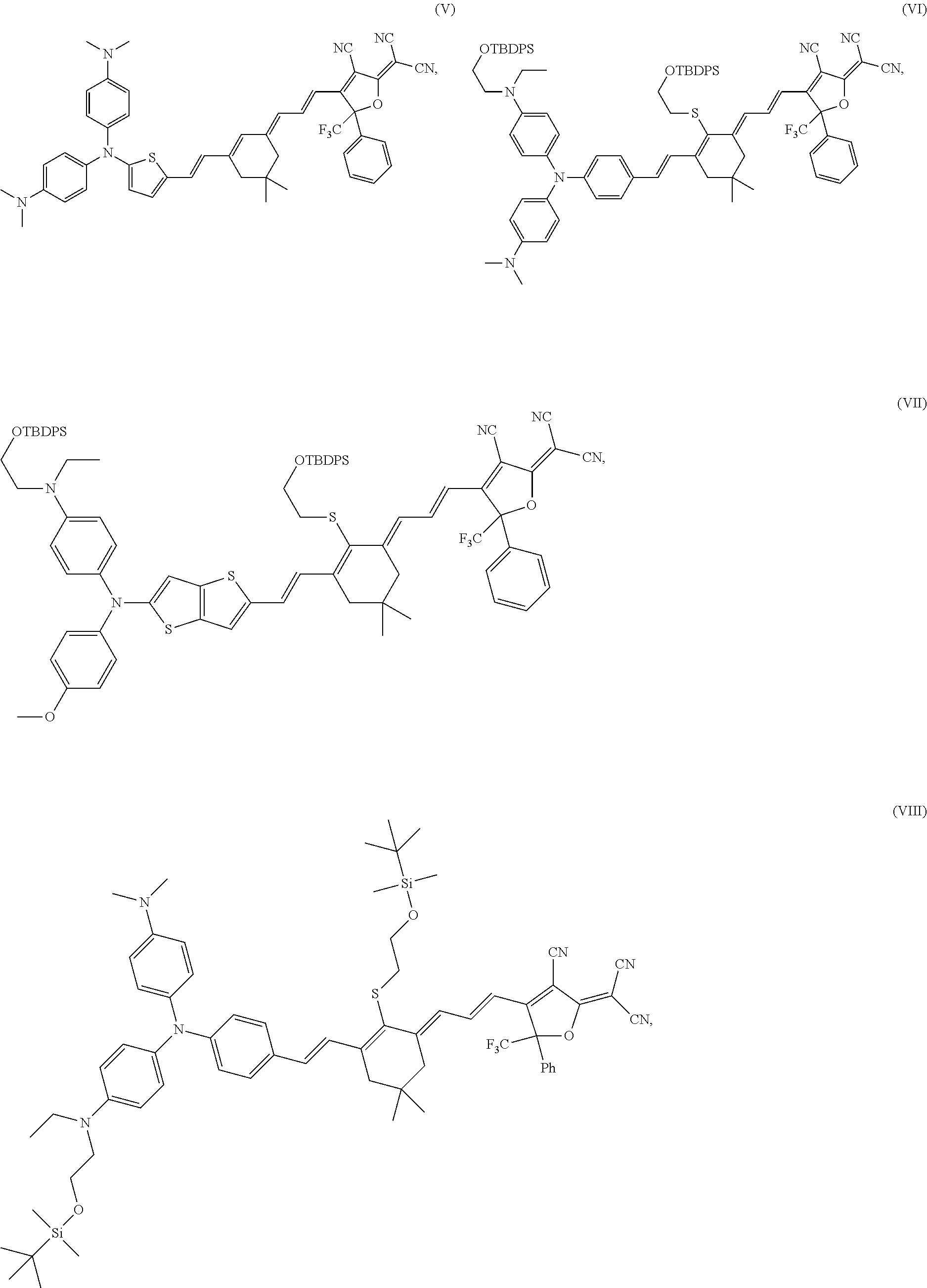 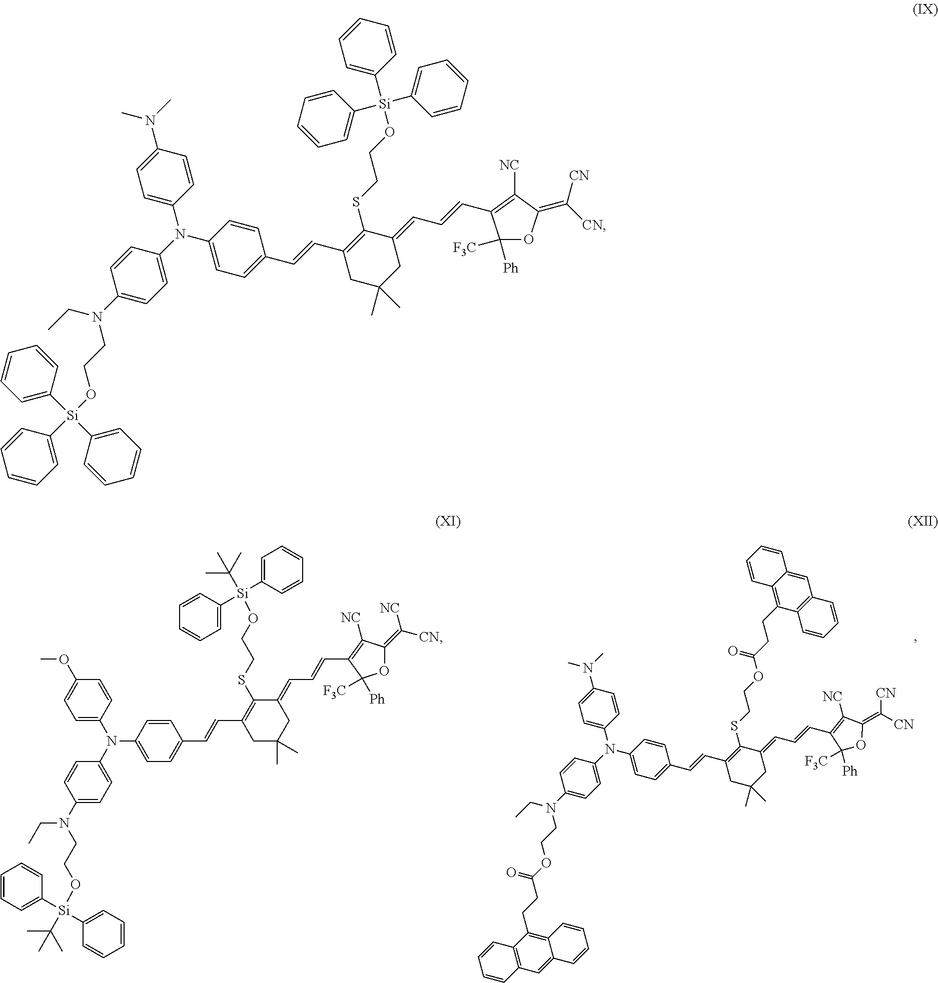 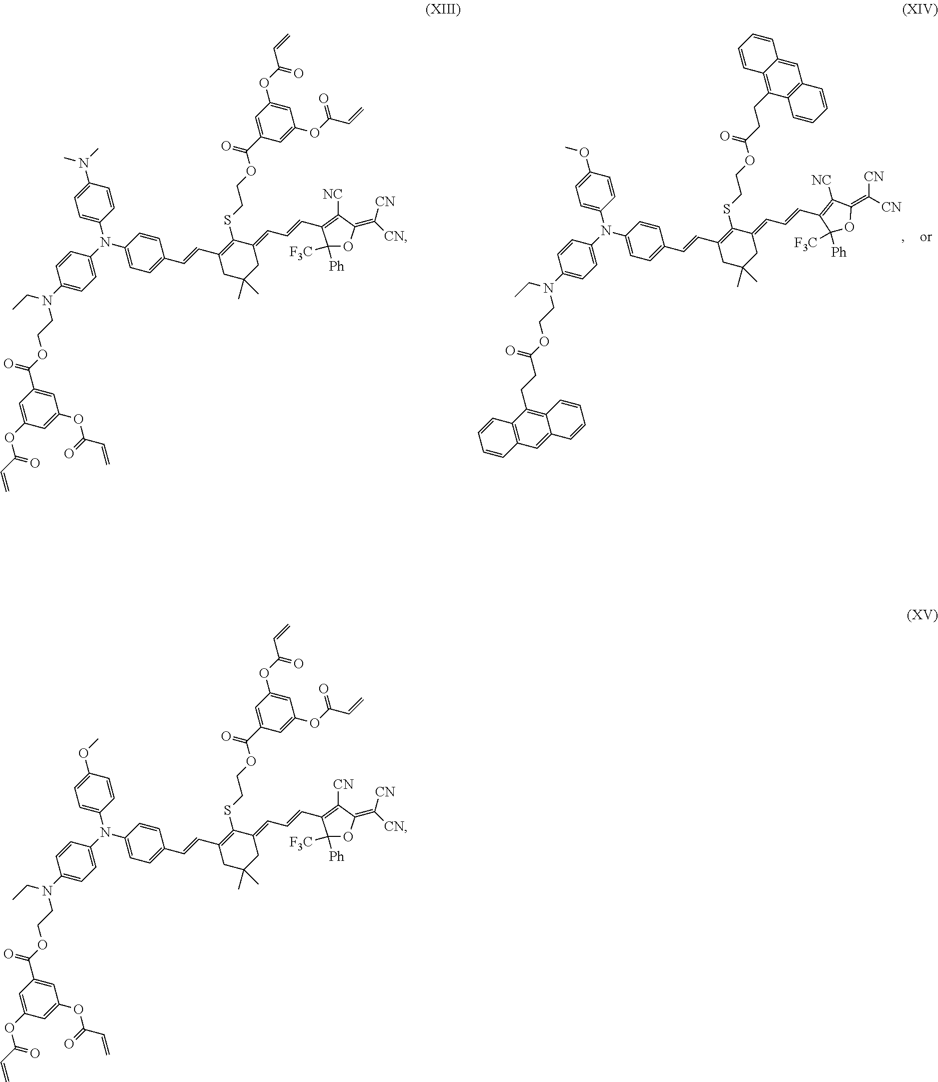 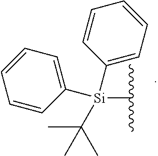 [SMILES: *[Si](c1ccccc1)(c1ccccc1)C(C)(C)C]
wherein TBDPS is

14. A film having electro-optic activity comprising one or more compounds of claim 1.

15. The film of claim 14, wherein the film further comprises a polymer.

16. The film of claim 14, wherein the film has a T_{g} of about 105° C. or greater.

17. A method for forming a film having electro-optic activity, comprising depositing a compound or mixture containing a compound of claim 1 on a substrate to provide a film, applying an aligning force to the film at a temperature sufficient to provide a film having at least a portion of the compounds aligned, and reducing the temperature of the film to provide a film having electro-optic activity.

18. An electro-optic device comprising a compound of claim 1.

19. An electro-optic device comprising a film of claim 14.

20. The electro-optic device of claim 19, wherein the device is an electro-optic modulator, antenna, Mach-Zehnder modulator, phase modulator, silicon-organic hybrid modulator, plasmonic-organic hybrid modulator, electrical-to-optical convertor, terahertz detector, frequency shifter, or frequency comb source.
